(19) United States
(12) Patent Application Publication
(10) Pub. No.: US 2007/0042411 A1
(43) Pub. Date: Feb. 22, 2007

(54) Multiplexed PCR assay for detection of bovine spongiform encephalopathy (BSE)-associated bovine prion protein gene (PRNP) polymorphisms

(75) Inventors: Christopher M. Seabury, College Station, TX (US); James N. Derr, College Station, TX (US)

Correspondence Address: HOWREY LLP, C/O IP DOCKETING DEPARTMENT, 2941 FAIRVIEW PARK DRIVE, SUITE 200, FALLS CHURCH, VA, 22042-7195, US

(73) Assignee: The Texas A&M University System

(21) Appl. No.: 11/505,583

(22) Filed: Aug. 17, 2006

Related U.S. Application Data
(60) Provisional application No. 60/708,958, filed on Aug. 17, 2005.

(51) Int. Cl. C12Q 1/68; C12P 19/34; C12M 1/34

(52) U.S. Cl. 435/6; 435/91.2; 435/287.2

(57) ABSTRACT

A multiplexed PCR assay system and method are provided for detection of bovine prion protein gene (PRNP) insertion/deletion polymorphisms that are associated with bovine spongiform encephalopathy (BSE), thereby identifying a population of cattle or individual therefrom that possesses an elevated level of putative resistance or susceptibility to BSE. Such system or method uses a mixture of primers targeting PRNP insertion/deletion polymorphisms in the putative promoter, intron 1, and 3′ untranslated region of the bovine PRNP gene. Also provided is a kit for identifying a population of cattle or individual therefrom that possesses an elevated level of putative resistance or susceptibility to BSE.

This application claims the benefit under 35 U.S.C. 119(e) of U.S. Provisional Application Ser. No. 60/708,958, filed Aug. 17, 2005, the entire contents of which are incorporated herein by reference.

### BACKGROUND OF THE INVENTION

1. Field of the Invention

The present invention generally relates to molecular biology and animal pathology. More specifically, the present invention relates to a multiplexed PCR assay system and/or method for detection of bovine prion protein gene (PRNP) polymorphisms that are associated with bovine spongiform encephalopathy (BSE), thereby identifying a population of cattle or an individual therefrom that possesses an elevated level of putative resistance or susceptibility to BSE.

2. Description of the Related Art

Transmissible spongiform encephalopathies (TSEs), or prion diseases, are inevitably fatal neurodegenerative diseases that occur in a variety of mammalian species, including humans as well as domestic and wild animals, and are often characterized by dementia and/or ataxia. (Collinge, 2001; Prusiner, 1998.) The pathogenic agents of prion diseases are infectious, protease-resistant proteins which arise through modification of the host-encoded normal cellular prion protein (PrP^{C}). Moreover, prion diseases may occur as genetic, infectious, or sporadic disorders. Additionally, while no definitive consensus regarding the precise function of PrP^{C} has been reached to date (Aguzzi and Hardt, 2003; Collinge, 2001), it has been suggested to promote synaptic homeostasis (Collinge et al., 1994), aid in neurite outgrowth and neuronal survival (Chen et al., 2003), and function as a cell-surface receptor for signal transduction (Mouillet-Richard et al., 2000).

Bovine spongiform encephalopathy (BSE), also known as mad cow disease, resulting from ingestion of scrapie and/or BSE infected meat and bone meal, has also been implicated in the development of variant Creutzfeld-Jakob disease (vCJD) in humans via consumption of beef from BSE-affected cattle. (Bruce et al., 1997; Scott et al., 1999; Collinge, 2001; Asante et al., 2002.) To date, several nonsynonymous single nucleotide polymorphisms (SNPs) within the human and ovine prion protein gene (PRNP) have been associated with resistance or susceptibility to prion diseases (Belt et al., 1995; Collinge, 2001; Baylis et al., 2002), and marker-assisted selection programs aimed at enriching the frequency of resistant ovine PRNP alleles in Dutch and British sheep has ensued. (Schreuder et al., 1997; Arnold et al., 2002.) Additionally, a novel association was recently documented between BSE susceptibility and specific bovine PRNP insertion/deletion polymorphisms within the putative promoter as well as intron 1 of a few German cattle breeds, supporting the hypothesis that mutations potentially influencing the level of bovine PRNP expression might also influence incubation time and susceptibility to BSE. (Bossers et al., 1996; Sander et al., 2004.) Sander et al. (2005) has recently associated certain genomic regions and relevant polymorphisms with either increased or decreased levels of bovine PRNP gene expression. Moreover, the incubation period of TSE's in transgenic mice expressing either bovine or ovine PRNP is inversely proportional to the amount of prion protein produced in the brains of the mice (Prusiner et al. 1990; Vilotte et al. 2001; Castilla et al. 2004.)

There is a need for an effective and precise system and/or method for rapidly and economically detecting bovine PRNP insertion/deletion polymorphisms that are associated with the expression of BSE, thereby identifying a population of cattle or an individual therefrom that possesses an elevated level of putative resistance or susceptibility to BSE.

### SUMMARY OF THE INVENTION

The present invention is directed to a system for detecting bovine PRNP insertion/deletion polymorphisms that are associated with bovine spongiform encephalopathy (BSE). Also provided is a method for identifying a population of cattle or an individual therefrom that possesses an elevated level of putative resistance or susceptibility to BSE. A kit for rapidly identifying a population of cattle or an individual therefrom that possesses an elevated level of putative resistance or susceptibility to BSE is further provided.

In one embodiment of the present invention, there is provided a multiplexed PCR system for identifying a population of cattle or an individual therefrom that possesses an elevated level of putative resistance or susceptibility to bovine spongiform encephalopathy (BSE), comprising a mixture of primers targeting bovine prion protein gene (PRNP) insertion/deletion polymorphisms in the putative promoter, intron 1, and 3′ untranslated region of the bovine PRNP gene, wherein one or more of the targeted PRNP insertion/deletion polymorphisms are associated with BSE.

In another embodiment of the present invention, there is provided a method for identifying an individual cow, bull or calf that possesses an elevated level of putative resistance or susceptibility to bovine spongiform encephalopathy (BSE). This method comprises obtaining a DNA sample from the cow, bull or calf; and then amplifying the DNA sample using a mixture of primers targeting bovine prion protein gene (PRNP) insertion/deletion polymorphisms in the putative promoter, intron 1, and 3′ untranslated region of the bovine PRNP gene, wherein one or more of the targeted PRNP insertion/deletion polymorphisms are associated with BSE. As a result, the presence of amplified DNA product of the bovine PRNP putative promoter, intron 1, and 3′ untranslated region corresponding to the one or more of the PRNP insertion polymorphisms indicates that the cow, bull or calf possesses an elevated level of putative resistance to BSE, whereas the presence of amplified DNA product of the bovine PRNP putative promoter, intron 1, and 3′ untranslated region corresponding to the one or more of the PRNP deletion polymorphisms indicates that the cow, bull or calf possesses an elevated level of putative susceptibility to BSE.

In still another embodiment of the present invention, there is a method for identifying a population of cattle that possesses an elevated level of putative resistance or susceptibility to bovine spongiform encephalopathy (BSE) collectively. This method comprises the steps of obtaining DNA samples from the population of cattle; amplifying each of the DNA samples using a mixture of primers targeting bovine prion protein gene (PRNP) insertion/deletion polymorphisms in the putative promoter, intron 1, and 3′ untranslated region of the bovine PRNP gene, wherein one or more of the PRNP insertion/deletion polymorphisms are associated with BSE; detecting amplified DNA product of the bovine PRNP putative promoter, intron 1, and 3′ untranslated region corresponding to the one or more of the PRNP insertion/deletion polymorphisms; and determining the frequency of the one or more of the PRNP insertion/deletion polymorphisms. If a significantly higher frequency of the one or more of the PRNP insertion polymorphisms exists in the DNA samples overall than observed in a reference cattle population affected by BSE, the population of cattle under evaluation is considered to possess an elevated level of putative resistance to BSE. If no significant differences exist between the frequency of the one or more of the PRNP deletion polymorphisms in the DNA samples and that observed in a reference cattle population affected by BSE, the population of cattle under evaluation is considered to possess an elevated level of putative susceptibility to BSE.

In still yet another embodiment of the present invention, there is provided a kit for identifying a population of cattle or an individual therefrom that possesses an elevated level of putative resistance or susceptibility to bovine spongiform encephalopathy, which kit comprises, in a suitable container, a mixture of PCR primers targeting bovine prion protein gene (PRNP) insertion/deletion polymorphisms in the putative promoter, intron 1, and 3′ untranslated region of the bovine PRNP gene, wherein one or more of the targeted PRNP insertion/deletion polymorphisms are associated with BSE.

The foregoing and other advantages of the present invention will be apparent to those skilled in the art, in view of the following detailed description of the preferred embodiment of the present invention, taken in conjunction with the accompanying drawings.

### BRIEF DESCRIPTION OF THE DRAWINGS

Features of the present invention as well as a preferred mode of use, further objectives, and advantages thereof, will best be understood by reference to the following detailed description of an illustrative embodiment when read in conjunction with the accompanying drawing, wherein:

FIG. 1 is a graphical depiction of the multiplexed PCR products corresponding to insertion/deletion polymorphisms in the bovine PRNP putative promoter, intron 1, and 3′ untranslated region (UTR) in accordance with the present invention.

### DETAILED DESCRIPTION

In order to provide a clear and consistent understanding of the specification and claims, including the scope given to such terms, the following definitions are provided:

As used herein, the term “putative resistance to bovine spongiform encephalopathy” refers to a tangible level and/or degree of innate resistance to bovine spongiform encephalopathy.

As used herein, the term “putative susceptibility to bovine spongiform encephalopathy” refers to a tangible level and/or degree of innate susceptibility to bovine spongiform encephalopathy.

As used herein, the term “an elevated level of putative resistance to bovine spongiform encephalopathy” refers to any advanced level, state, degree, or magnitude of innate resistance to bovine spongiform encephalopathy that is tangible, apparent, and/or detectable via statistical analysis, experimentation, and/or the prior art.

As used herein, the term “an elevated level of putative susceptibility to bovine spongiform encephalopathy” refers to any advanced level, state, degree, or magnitude of innate susceptibility to bovine spongiform encephalopathy that is tangible, apparent, and/or detectable via statistical analysis, experimentation, and/or the prior art.

As used herein, the term “multiplexed PCR assay” refers to a PCR assay which targets multiple genetic loci of DNA of interest simultaneously.

As used herein, the term “marker-assisted selection program” refers to a selective breeding and/or culling program enabled via the multiplexed PCR assay provided herein whereby cattle are identified, separated and selected based on genetic information generated for the bovine PRNP promoter, intron 1, and 3′UTR as well as the relationship between this genetic variation and putative resistance or susceptibility to bovine spongiform encephalopathy (BSE).

As used herein, the term “bovine PRNP insertion/deletion polymorphism” refers to naturally occurring genetic variation within the bovine PRNP gene consisting of insertions and deletions of nucleotides.

As used herein, the term “allelic distribution of bovine PRNP insertion/deletion polymorphism” or “allelic frequency for bovine PRNP insertion/deletion polymorphism” refers to frequencies (proportions) of the insertion/deletion alleles observed at multiple loci, e.g., in the putative promoter, intron 1, and 3′ UTR of the bovine PRNP gene.

As used herein, the term “genotypic distribution of bovine PRNP insertion/deletion polymorphism” or “genotypic frequency for bovine PRNP insertion/deletion polymorphism” refers to frequencies (proportions) of bovine PRNP genotypes observed in a population of cattle, which genotypes contain insertion/deletion polymorphisms at multiple loci, e.g. in the putative promoter, intron 1, and 3′UTR regions of the bovine PRNP gene.

As used herein, the term “distribution of BSE-associated PRNP alleles” refers to observed frequencies (proportions) of PRNP alleles that were previously identified to be statistically associated with susceptibility to bovine spongiform encephalopathy (BSE).

As used herein, the term “distribution of BSE-associated PRNP genotypes” refers to observed frequencies (proportions) of PRNP genotypes that were previously identified to be statistically associated with susceptibility to bovine spongiform encephalopathy (BSE).

As used herein, the term “PRNP genotyping” refers to collection of diploid polymorphism data from the bovine PRNP gene using the multiplexed PCR assay described herein.

The present invention is directed to a multiplexed PCR assay system and/or method for detecting bovine prion protein gene (PRNP) polymorphisms that are associated with bovine spongiform encephalopathy (BSE), thereby identifying a population of cattle or an individual therefrom that possesses an elevated level of putative resistance or susceptibility to BSE. The present invention is further directed to a kit for identifying a population of cattle or an individual therefrom that possesses an elevated level of putative resistance or susceptibility to BSE.

In one embodiment of the present invention, there is provided a multiplexed PCR system for identifying a population of cattle or an individual therefrom that possesses an elevated level of putative resistance or susceptibility to bovine spongiform encephalopathy (BSE), comprising a mixture of primers targeting bovine prion protein gene (PRNP) insertion/deletion polymorphisms in the putative promoter, intron 1, and 3′ untranslated region of the bovine PRNP gene, wherein one or more of the targeted PRNP insertion/deletion polymorphisms are associated with BSE. Preferably, the one or more of the targeted PRNP insertion/deletion polymorphisms comprise the 23 bp insertion/deletion in the putative promoter, 12 bp insertion/deletion in intron 1, or 14 bp insertion/deletion in the 3′ untranslated region of the bovine PRNP gene.

Preferably, that multiplexed PCR system amplifies DNA of the putative promoter, intron 1, and 3′ untranslated region of the bovine PRNP gene simultaneously in a single reaction. Also, the primers used are preferably labeled, and more preferably, fluorescently labeled.

In another embodiment of the present invention, there is provided a method for identifying an individual cow, bull or calf that possesses an elevated level of putative resistance or susceptibility to bovine spongiform encephalopathy (BSE). This method comprises obtaining a DNA sample from the cow, bull or calf; and then amplifying the DNA sample using a mixture of primers targeting bovine prion protein gene (PRNP) insertion/deletion polymorphisms in the putative promoter, intron 1, and 3′ untranslated region of the bovine PRNP gene, wherein one or more of the targeted PRNP insertion/deletion polymorphisms are associated with BSE. As a result, the presence of amplified DNA product of the bovine PRNP putative promoter, intron 1, and to a lesser extent, the 3′ untranslated region corresponding to the one or more of the PRNP insertion polymorphisms indicates that the cow, bull or calf possesses an elevated level of putative resistance to BSE, whereas the presence of amplified DNA product of the bovine PRNP putative promoter, intron 1, and to a lesser extent, the 3′ untranslated region corresponding to the one or more of the PRNP deletion polymorphisms indicates that the cow, bull or calf possesses an elevated level of putative susceptibility to BSE.

Preferably, the one or more of the targeted PRNP insertion/deletion polymorphisms comprise the 23 bp insertion/deletion in the putative promoter, 12 bp insertion/deletion in intron 1, or 14 bp insertion/deletion in the 3′ untranslated region of the bovine PRNP gene. More preferably, the one or more of the targeted PRNP insertion/deletion polymorphisms that are associated with BSE comprise the 23 bp insertion/deletion in the putative promoter or 12 bp insertion/deletion in intron 1. Also, the primers used are preferably labeled, and more preferably, fluorescently labeled.

In still another embodiment of the present invention, there is a method for identifying a population of cattle that possesses an elevated level of putative resistance or susceptibility to bovine spongiform encephalopathy (BSE). This method comprises the steps of obtaining DNA samples from the population of cattle; amplifying each of the DNA samples using a mixture of primers targeting bovine prion protein gene (PRNP) insertion/deletion polymorphisms in the putative promoter, intron 1, and 3′ untranslated region of the bovine PRNP gene, wherein one or more of the targeted PRNP insertion/deletion polymorphisms are associated with BSE; detecting amplified DNA product of the bovine PRNP putative promoter, intron 1, and 3′ untranslated region corresponding to the one or more of the PRNP insertion/deletion polymorphisms; and determining the frequency of the one or more of the PRNP insertion/deletion polymorphisms. If a significantly higher frequency of the one or more of the PRNP insertion polymorphisms, particularly with respect to the putative promoter and intron 1 insertion polymorphisms, exists in the tested DNA samples as compared to a reference cattle population affected by BSE, the population of cattle under evaluation is considered to possess an elevated level of putative resistance to BSE. If no significant differences exist between the frequency of the one or more of the PRNP deletion polymorphisms in the tested DNA samples and that in a reference cattle population affected by BSE, particularly with respect to the putative promoter and intron 1 deletion polymorphisms, the population of cattle under evaluation is considered to possess an elevated level of putative susceptibility to BSE.

In still yet another embodiment of the present invention, there is provided a kit for identifying a population of cattle or an individual therefrom that possesses an elevated level of putative resistance or susceptibility to bovine spongiform encephalopathy, which kit comprises, in a suitable container, a mixture of PCR primers targeting bovine prion protein gene (PRNP) insertion/deletion polymorphisms in the putative promoter, intron 1, and 3′ untranslated region of the bovine PRNP gene, wherein one or more of the targeted PRNP insertion/deletion polymorphisms are associated with BSE.

Preferably, the one or more of the targeted PRNP insertion/deletion polymorphisms comprise, more particularly, the 23 bp insertion/deletion in the putative promoter, or 12 bp insertion/deletion in intron 1, or to a lesser extent, 14 bp insertion/deletion in the 3′ untranslated region of the bovine PRNP gene. Also, the primers used are preferably labeled, and more preferably, fluorescently labeled.

In a preferred embodiment, the present system/method provides large-scale, rapid detection of PRNP insertion/deletion polymorphisms, or large-scale, rapid identification of a population of cattle or an individual therefrom putatively resistant or susceptible to bovine spongiform encephalopathy enabled by the present multiplexed PCR assay. The present system, in combination with modern laboratory equipment and reasonably skilled personnel, has the potential to facilitate analysis for thousands of viable cattle DNA samples within a 48-hour period.

Also in a preferred embodiment, the primers can be fluorescently labeled. Various fluorescent dye labels can be used, for example, 2′-chloro-5′-fluoro-7′,8′-fused phenyl-1,4-dichloro-6-carboxyfluorescein (NED) (Applied Biosystems, Foster City, Calif.), 6-carboxyfluorescein (6-FAM) (Sigma-Genosys, The Woodlands, Tex.), or 2′,4′,5′,7′,1,4-hexachlorofluorescein (HEX) (Sigma-Genosys, The Woodlands, Tex.).

The following example is given for the purpose of illustrating various embodiments of the invention and is not meant to limit the present invention in any fashion.

### EXAMPLE

Materials

Bovine PRNP insertion/deletion polymorphisms within the putative promoter, intron 1, and the 3′ UTR that are known to be associated with bovine spongiform encephalopathy (BSE) are given in GenBank AJ298878 and GenBank BN000291 documents. Specifically, the 23 bp (tctcagatgtcttcccaacagca; SEQ ID NO:2) insertion/deletion polymorphism in the putative promoter of the bovine PRNP gene occurs between the nucleotides 47836 and 47837 of the bovine PRNP gene (SEQ ID NO:1); the 12 bp (gggggccgcggc; SEQ ID NO:3) insertion/deletion polymorphism in intron 1 of the bovine PRNP gene occurs between the nucleotides 49729 and 49730 of the bovine PRNP gene (SEQ ID NO:1); and the 14 bp (tttttgtaaggtac; SEQ ID NO:4) insertion/deletion polymorphism in the 3′ UTR of the bovine PRNP gene occurs between the nucleotides 68019 and 68032 of the bovine PRNP gene (SEQ ID NO:1).

To evaluate the frequencies of bovine PRNP insertion/deletion polymorphisms within the putative promoter, intron 1, and the 3′ UTR in a cattle population, a DNA panel was utilized consisting of 132 artificial insemination (Al) sires from 39 domestic cattle breeds. The source of DNA was spernatozoa purchased through commercial dealers. Names of breeds and sample sizes (n) were as follows: Black Angus (4); Beefalo (1); Beefmaster (5); Belgian Blue (4); Blonde D'Aquitaine (5); Braford (4); Brahman (4); Brahmousin (2); Brangus (5); Braunvieh (5); Brown Swiss (4); Charolais (5); Chianina-Chiangus (5); Corriente (1); Gelbvieh (4); Hereford (3); Holstein (4); Jersey (1); Limousin (3); Maine Anjou (4); Murray Grey (2); Nelore (8); Normande (1); Piedmontese (2); Pinzgauer (1); Red Angus (4); Red Brangus (2); Red Poll (1); Romagnola (2); Salers (3); Santa Gertrudis (4); Scottish Highland (1); Senepol (2); Shorthorn (5); Simbrah (3); Simmental (8); Tarentaise (1); Texas Longhorn (4); Three-way-cross (4); White Park (1). Six of the Nelore were not AI sires. Care was taken to select unrelated sires.

Multiplexed PCR Assay and Validation Techniques

Forward and reverse primer pairs targeting known insertion/deletion polymorphisms in the bovine PRNP putative promoter (23 bp insertion/deletion) (Sander et al., 2004), intron 1 (12 bp insertion/deletion) (Hills et al., 2001), and the 3′ UTR (14 bp insertion/deletion) (Hills et al., 2003) were utilized together in single multiplexed 5 μl volume reactions. Specifically, the forward and reverse primers used targeting the putative promoter polymorphism were PRNP47784F (gtgccagccatgtaagtg; SEQ ID NO:5) and PRNP47883R (tggacaggcacaatggg; SEQ ID NO:6); the forward and reverse primers used targeting the intron 1 polymorphism were PRNP49686F (ttaccctcctggttaggag; SEQ ID NO:7) and PRNP49777R (ctagattcctacacaccac; SEQ ID NO:8); and the forward and reverse primers used targeting the 3′ UTR polymorphism were PRNP67976F (ctgttagagcaattaacatctg; SEQ ID NO:9) and PRNP68070R (ctagggattaaacccacgtc; SEQ ID NO:10).

Forward primers, respectively, were synthesized with 5′ fluorescent labels as follows: PRNP47784F 5′ NED (Applied Biosystems, Foster City, Calif.); PRNP49686F 5′ 6-FAM (Sigma-Genosys, The Woodlands, Tex.); PRNP67976F 5′ HEX (Sigma-Genosys, The Woodlands, Tex.).

All multiplexed PCR reactions were carried out on GeneAmp 9700 PCR Systems (Applied Biosystems). A PCR method was optimized for the multiplexed reactions, wherein the annealing temperature for the initial cycles is reduced by 1 Celsius degree (° C.) per thermal cycle, beginning from a high stringent starting temperature and ending several Celsius degrees lower, to enable optimization and specificity without experimental determination of a single optimized annealing temperature. After cycling through the annealing temperature step down stage, the remaining PCR cycles proceed at the lowest temperature until completion.

Preferably, the PCR reaction consisted of the following: 50-100 ng DNA, 0.375 units Taq polymerase (Promega, Madison, Wis.), 0.64 μM PRNP47784F-NED, 0.64 μM PRNP47883R, 0.64 μM PRNP49686F-6-FAM, 0.64 μM PRNP49777R, 0.52 μM PRNP67976F-HEX, 0.52 μM PRNP68070R, 3 mM MgCl₂ (Promega), 500 μM dNTPs (Promega), 1× MasterAmp™ PCR Enhancer (Epicentre, Madison, Wis.), and 1.5× Reaction Buffer (Promega). Optimized thermal cycling parameters were as follows: 2 min at 96° C.; 4 cycles×30 s at 96° C., 30 s at 58° C. (−1° C./cycle), 90 s at 65° C.; 31 cycles×30 s at 96° C., 30 s at 54° C., 90 s at 65° C.; 15 min at 65° C. Multiplexed PCR products were separated and analyzed on an ABI 3100 and/or 310 Genetic Analyzer (Applied Biosystems) and sized relative to an internal size standard (MAPMARKER LOW®, Bioventures). For comparison of consistency and validation of the multiplex assay developed, 10 sires were also genotyped via agarose gel electrophoresis following the methods of Sander et al. (2004).

Statistical Analysis

The distributions of PRNP alleles between all German cattle (a pool of healthy +BSE-affected cattle; Sander et al., 2004) and U. S. cattle sires, healthy German cattle and U. S. cattle sires, as well as BSE-affected German cattle and U. S. cattle sires were tested for significant differences using Fisher's exact test within the program STAT-SAK (G. E. Dallal; freeware) In addition, tests of genic and genotypic differentiation between the aforementioned groups were carried out using the G-based exact test of Goudet et al. (1996) within the program GENEPOP 3.1d (Raymond and Rousset, 1995) using the default parameters. In all cases, P<0.05 was considered statistically significant.

Results

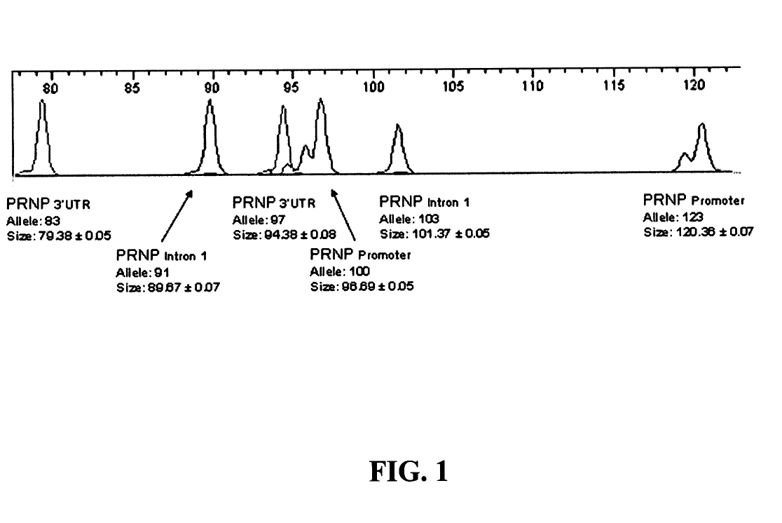

The multiplexed PCR assay developed for the rapid genotyping of BSE-associated bovine PRNP insertion/deletion polymorphisms in the putative promoter and intron 1 of the bovine PRNP gene, as well as known insertion/deletion polymorphisms within the 3′ UTR, is illustrated in FIG. 1. No significant differences in the distributions of bovine PRNP alleles and/or genotypes corresponding to the putative promoter, intron 1, and 3′ UTR were noted between German cattle as a whole (a pool of healthy +BSE-affected cattle) and the panel of U.S. cattle sires. However, significant differences in the allelic and genotypic distributions of bovine PRNP insertion/deletion polymorphisms in the putative promoter and intron 1 were noted when U. S. cattle sires were compared to healthy as well as BSE-affected German cattle (Table 1). Specifically, significant differences were detected between the distributions of PRNP putative promoter alleles for healthy German cattle and a panel of U.S. cattle sires (Table 1). Moreover, the frequency of the 23 bp promoter allele observed for the panel of U. S. cattle sires strongly resembled that previously reported for BSE-affected German cattle. (Sander et al. 2004.)

Table 1 demonstrates allelic and genotypic frequencies observed for bovine PRNP putative promoter, intron 1, and 3′ UTR insertion/deletion polymorphisms in healthy and BSE-affected German cattle (Sander et al., 2004) and a panel of commercial U. S. sires. Differences in the allelic and genotypic frequencies among the above-mentioned cattle populations were evaluated. Corresponding probabilities are shown in the last 3 columns with genic and genotypic differentiation above and below the diagonal, respectively (P<0.05 depicted in bold).

TABLE 1

| | | | Allele | | Genotype | | | | | |
|---|---|---|---|---|---|---|---|---|---|---|
| Locus | | (n) | + | − | + + | + − | − − | Healthy^{a} | Affected^{b} | U.S. sires |
| Promoter | Healthy^{a} | 48 | 0.43 | 0.57 | 0.21 | 0.44 | 0.35 | — | 0.0296^{c}; 0.0316^{d} | 0.0321^{c}; 0.0340^{d} |
| 23 bp | Affected^{b} | 43 | 0.27 | 0.73 | 0.05 | 0.44 | 0.51 | 0.0330^{e} | — | 0.5871^{c}; 0.5895^{d} |
| insertion/ | U.S. Sires | 132 | 0.30 | 0.70 | 0.14 | 0.32 | 0.54 | 0.0510^{e} | 0.6141^{e} | — |
| deletion | | | | | | | | | | |
| Intron 1 | Healthy^{a} | 48 | 0.49 | 0.51 | 0.21 | 0.56 | 0.23 | — | 0.0344^{c}; 0.0355^{d} | 1.0000^{c}; 1.0000^{d} |
| 12 bp | Affected^{b} | 43 | 0.33 | 0.67 | 0.09 | 0.47 | 0.44 | 0.0294^{e} | — | 0.0086^{c}; 0.0092^{d} |
| insertion/ | U.S. Sires | 132 | 0.49 | 0.51 | 0.32 | 0.35 | 0.33 | 1.0000^{e} | 0.0198^{e} | — |
| deletion | | | | | | | | | | |
| 3′ UTR^{f} | Healthy^{a} | 48 | 0.95 | 0.05 | 0.90 | 0.10 | 0.00 | — | 0.5530^{c}; 0.5565^{d} | 1.0000^{c}; 1.0000^{d} |
| 14 bp | Affected^{b} | 43 | 0.92 | 0.08 | 0.86 | 0.12 | 0.02 | 0.5773^{e} | — | 0.4632^{c}; 0.4661^{d} |
| insertion/ | U.S. Sires | 132 | 0.94 | 0.06 | 0.89 | 0.11 | 0.00^{g} | 0.8137^{e} | 0.6366^{e} | — |
| deletion | | | | | | | | | | |

^{a}Healthy German cattle and corresponding data from Sander et al. (2004).
^{b}BSE affected German cattle and corresponding data from Sander et al. (2004).
^{c}Probability obtained from Fisher's exact test in STAT-SAK (G. E. Dallal).
^{d}Probability obtained from Genic differentiation analysis using the G-based exact test of Goudet et al. (1996).
^{e}Probability obtained from Genotypic differentiation analysis using the G-based exact test of Goudet et al. (1996).
^{f}14 bp insertion/deletion consisting of 1 (−) or 2 (+) repeats
^{g}One sire out of 132 (actual observed frequency = 0.0075)

No significant difference was detected in the distribution of PRNP putative promoter genotypes between healthy German cattle and the panel of U. S. cattle sires. (P=0.0510; Table 1.) In contrast, the frequency of the 12 bp intron 1 allele observed for the panel of U. S. cattle sires was identical to that previously reported for healthy German cattle (Sander et al. 2004.) (P=1.0000; Table 1.) Significant differences were detected between the distributions of intron 1 alleles and genotypes for BSE-affected German cattle and the panel of U. S. cattle sires. (P=0.0198; Table 1.) No significant differences were detected in the distributions of alleles or genotypes corresponding to the 14 bp insertion/deletion within the PRNP 3′ UTR for the healthy German cattle and the panel of U. S. cattle sires, and the frequency of the 14 bp (+) 3′ UTR allele and corresponding++genotype for the panel of U. S. cattle sires was comparable to that reported for healthy German cattle (Sander et al., 2004.)

Observed PRNP insertion/deletion allele and genotype frequencies, subdivided by domestic breed for those breeds where three or more sires were sampled, are depicted in Table 2.

TABLE 2

| | | Promoter | | Intron 1 | | 3′ UTR | |
|---|---|---|---|---|---|---|---|
| | | 23 bp | | 12 bp | | 14 bp | |
| | | insertion/ | | insertion/ | | insertion/ | |
| Domestic | | deletion | | deletion | | deletion | |
| Cattle Breed ^{a} | (n) | + | − | + | − | + | − |
| Angus ^{b} | 8 | 0.188 | 0.812 | 0.188 | 0.812 | 1.000 | 0.000 |
| Beefmaster | 5 | 0.000 | 1.000 | 0.200 | 0.800 | 1.000 | 0.000 |
| Belgian Blue | 4 | 0.250 | 0.750 | 0.250 | 0.750 | 1.000 | 0.000 |
| Blonde d′ ^{c} | 5 | 0.500 | 0.500 | 0.600 | 0.400 | 0.900 | 0.100 |
| Braford | 4 | 0.125 | 0.875 | 0.250 | 0.750 | 0.500 | 0.500 |
| Brahman | 4 | 0.250 | 0.750 | 1.000 | 0.000 | 0.875 | 0.125 |
| Brangus ^{d} | 7 | 0.071 | 0.929 | 0.571 | 0.429 | 1.000 | 0.000 |
| Braunvieh | 5 | 0.400 | 0.600 | 0.700 | 0.300 | 0.900 | 0.100 |
| Brown Swiss | 4 | 1.000 | 0.000 | 1.000 | 0.000 | 1.000 | 0.000 |
| Charolais | 5 | 0.600 | 0.400 | 0.700 | 0.300 | 1.000 | 0.000 |
| Chianina ^{e} | 5 | 0.500 | 0.500 | 0.500 | 0.500 | 0.900 | 0.100 |
| Gelbvieh | 4 | 0.250 | 0.750 | 0.375 | 0.625 | 1.000 | 0.000 |
| Hereford | 3 | 0.500 | 0.500 | 0.500 | 0.500 | 1.000 | 0.000 |
| Holstein | 4 | 0.375 | 0.625 | 0.375 | 0.625 | 1.000 | 0.000 |
| Limousin | 3 | 0.500 | 0.500 | 0.833 | 0.167 | 1.000 | 0.000 |
| Maine Anjou | 4 | 0.250 | 0.750 | 0.375 | 0.625 | 1.000 | 0.000 |
| Nelore | 8 | 0.000 | 1.000 | 1.000 | 0.000 | 1.000 | 0.000 |
| Santa Gertrudis | 8 | 0.000 | 1.000 | 0.375 | 0.625 | 1.000 | 0.000 |
| Shorthorn | 5 | 0.100 | 0.900 | 0.100 | 0.900 | 1.000 | 0.000 |
| Simbrah | 3 | 0.333 | 0.667 | 0.833 | 0.167 | 0.833 | 0.167 |
| Simmental | 8 | 0.000 | 1.000 | 0.000 | 1.000 | 0.875 | 0.125 |
| TX Longhorn | 4 | 0.625 | 0.375 | 0.750 | 0.250 | 1.000 | 0.000 |

^{a} Allele frequencies depicted herein are not intended to represent entire breeds
^{b} Black and Red Angus combined
^{c} Blonde d′ Aquitaine
^{d} Black and Red Brangus combined
^{e} Chianina/Chiangus (Chianina × Angus)

Discussion

Unlike previous bovine PRNP studies involving U. S. cattle (Ryan and Womack, 1993; Neibergs et al., 1994; Heaton et al., 2003), the present study provides the first comprehensive survey of bovine PRNP insertion/deletion polymorphisms corresponding to the putative promoter, intron 1, and 3′ UTR, facilitated through the utilization of a large and diverse panel of commercial U. S. Al sires from 39 domestic breeds.

While PRNP polymorphisms associated with resistance to prion diseases have previously been described for humans, sheep, and goats (Belt et al., 1995; Collinge, 2001; Billinis et al., 2002), a similar association has only recently been extended to domestic cattle (Sander et al., 2004; Sander et al., 2005). The results of Sander et al. (2004; 2005) clearly demonstrate that domestic cattle possess PRNP alleles and genotypes significantly associated with susceptibility to BSE. Therefore, an assay capable of the rapid identification of individual cows, bulls or calves, and/or a population of cattle possessing PRNP genotypes significantly associated with the phenotypic expression of BSE is potentially invaluable to both the domestic cattle industry as well as human health. The present study provides such an assay based on data generated for healthy and BSE-affected German cattle by Sander et al. (2004; 2005) (FIG. 1.) However, it should be noted that data generated for any BSE-affected cattle population may be used for comparison or reference when assessing the capability of the present multiplexed PCR assay in identifying a population of cattle or individual therefrom that possesses an elevated level of putative resistance or susceptibility to BSE.

Additionally, the present study also provides, for the first time, a survey of the allelic and genotypic frequencies corresponding to PRNP insertion/deletion polymorphisms previously associated with susceptibility to BSE for specific domestic cattle breeds. (Table 2.) However, while care was taken to select several unrelated sires from each respective breed, some degree of caution remains necessary when interpreting the relationship between the observed frequencies depicted in Table 2 and overall putative susceptibility or resistance to BSE for each breed depicted.

The significance of the differences between PRNP allele and/or genotype distributions for a panel of U. S. cattle sires and German cattle was investigated. (Table 1.) Given that the newly described 23 bp bovine PRNP putative promoter insertion/deletion polymorphism exhibited the most significant association with BSE in German cattle, Sander et al. (2004) subsequently attributed the statistical significance noted between the 12 bp intron 1 insertion/deletion and BSE to tight linkage between the putative promoter and intron 1. However, the potential effects and/or role(s) of each polymorphism with respect to bovine PRNP expression and BSE susceptibility (Table 1) should be considered. Furthermore, it should be noted that the 12 bp intron 1 insertion/deletion was previously determined to lie within a region important for retention of full PRNP putative promoter activity in cultured bovine CKT-1 cells, and the 12 bp deletion was determined to remove a putative binding site for the transcription factor SPI (Inoue et al., 1997; Hills et al., 2001). Notably, the panel of U. S. cattle sires used in the present study possesses a significantly higher frequency of the 12 bp (+) intron 1 allele and corresponding ++genotype than BSE-affected German cattle (Table 1), but differs significantly from healthy German cattle with respect to the frequency of the 23 bp (+) promoter allele which previously exhibited the most significant association with BSE status. If these data are interpreted with strict adherence to the results of Sander et al. (2004), it appears that the panel of U. S. cattle sires, collectively, is largely susceptible to BSE based on polymorphism data generated for the bovine PRNP putative promoter.

The present study provides a single high-throughput optimized system for marker-assisted selection/identification of cattle putatively resistant to BSE, thereby providing a method of reduction of susceptible individuals in breeding stocks. Such a molecular platform for marker-assisted selection of resistant cattle was fully optimized in the present study to enable the simultaneous targeting of the three regions interrogated, that is, the putative promoter, intron 1, and 3′ untranslated region of the bovine PRNP gene. Compared to an archaic data collection approach that is commonly known in the art, the present multiplexed PCR assay offers advantages of a higher sensitivity, specificity and efficiency as well as being less expensive.

In addition, the present study provides the most comprehensive investigation into the frequencies of alleles and genotypes that are statistically associated with susceptibility to BSE in a variety of cattle breeds. Compared to 7 European cattle breeds addressed in Sander et al., some of which are not really present in the U.S., the present study provides allelic and genotypic data for 39 U.S. domestic breeds, the majority of which were never studied previously with respect to the bovine PRNP polymorphisms targeted. The present multiplexed system provides much more comprehensive data regarding the presence or absence of alleles/genotypes that confer tangible levels of resistance/susceptibility in specific breeds of cattle that are economically important in the U.S.

While the invention has been shown in only a few of its forms, it should be apparent to those skilled in the art that it is not so limited but susceptible to various changes without departing from the scope of the invention.

### BIBLIOGRAPHY

Aguzzi A, Hardt W-D (2003) Dangerous liaisons between a microbe and the prion protein. J Exp Med 198:1-4.
Arnold M, Meek C, Webb C R, Hoinville L J (2002) Assessing the efficacy of a ram-genotyping programme to reduce susceptibility to scrapie in Great Britain. Prev Vet Med 56, 227-249.
Asante E A, Linehan J M, Desbruslais M, Joiner S, Gowland I, Wood A L, Welch J, Hill A F, Lloyd S E, Wadsworth J D, Collinge J (2002) BSE prions propagate as either variant CJD-like or sporadic CJD-like prion strains in transgenic mice expressing human prion protein. EMBO J 21,6358-6366.
Baylis M, Houston F, Kao R R, McLean A R, Hunter N, Gravenor M B (2002) BSE-a wolf in sheep's clothing? Trends Microbiol 10, 563-570.
Belt P B, Muileman I H, Schreuder B E, Bos-de Ruijter J, Gielkens A L, Smits M A (1995) Identification of five allelic variants of the sheep PrP gene and their association with natural scrapie. J Gen Virol 76, 509-517.
Billinis C, Panagiotidis C H, Psychas V, Argyroudis S, Nicolaou A, Leontides S, Papadopoulos O, Sklaviadis T (2002) Prion protein gene polymorphisms in natural goat scrapie. J Gen Virol 83, 713-721.
Bossers A, Schreuder B E, Muileman I H, Belt P B, Smits M A (1996) PrP genotype contributes to determining survival times of sheep with natural scrapie. J Gen Virol 77, 2669-2673.
Bruce M E, Will R G, Ironside J W, McConnell I, Drummond D, Suttie A, McCardle L, Chree A, Hope J, Birkett C, Cousens S, Fraser H, Bostock C J (1997) Transmissions to mice indicate that ‘new variant’ CJD is caused by the BSE agent. Nature 389, 498-501.
Castilla J, Gutierrez-Adan A, Brun A, Pintado B, Parra B, et al. (2004) Different behavior toward bovine spongiform encephalopathy infection of bovine prion protein transgenic mice with one extra repeat octapeptide insert mutation. J Neurosci 24, 2156-2164.
Chen S, Mange A, Dong L, Lehmann S, Schachner M (2003) Prion protein as trans-interacting partner for neurons is involved in neurite outgrowth and neuronal survival. Mol Cell Neurosci 22, 227-233.
Collinge J (2001) Prion diseases of humans and animals: their causes and molecular basis. Ann Rev Neurosci 24, 519-50.
Collinge J, Whittington M A, Sidle K C, Smith C J, Palmer M S, Clarke A R, Jefferys J G (1994) Prion protein is necessary for normal synaptic function. Nature 370, 295-297.
Goudet J, Raymond M, De Meeüs T, Rousset F (1996) Testing differentiation in diploid populations. Genetics 144, 1933-1940.
Heaton M P, Leymaster K A, Freking B A, Hawk D A, Smith T P L, Keele J W, Snelling W M, Fox J M, Chitko-McKown C G, Laegreid W W (2003) Prion gene sequence variation within diverse groups of U. S. sheep, beef cattle, and deer. Mamm Genome 14, 765-777.
Hills D, Comincini S, Schlaepfer J, Dolf G, Ferretti L, Williams J L (2001) Complete genomic sequence of the bovine prion gene (PRNP) and polymorphism in its promoter region. Anim Genet 32, 231-232.
Hills D, Schlaepfer J, Comincini S, MacLean I, Dolf G, Ferretti L, Olsaker I, Williams J L (2003) Sequence variation in the bovine and ovine PRNP genes. Anim Genet 34, 183-190.
Inoue S, Tanaka M, Horiuchi M, Ishiguro N, Shinagawa M (1997) Characterization of the Bovine Prion Protein Gene: The Expression Requires Interaction between the Promoter and Intron. J Vet Med Sci 59, 175-183.
Mouillet-Richard S, Ermonval M, Chebassier C, Laplanche J L, Lehmann S, Launay J M, Kellerman O (2000) Signal transduction through prion protein. Science 289, 1925-28.
Neibergs H L, Ryan A M, Womack J E, Spooner R L, Williams J L (1994) Polymorphism analysis of the prion gene in BSE-affected and unaffected cattle. Anim Genet 25, 313-317.
Prusiner S B (1998) Prions. Proc Natl Acad Sci USA 95, 13363-13383.
Prusiner S B, Scott M, Foster D, Pan K M, Groth D, et al. (1990) Transgenic studies implicate interactions between homologous PrP isoforms in scrapie prion replication. Cell 63, 673-686.
Raymond M, Rousset F (1995) GENEPOP (Version 1.2): population genetics software for exact tests and ecumenicism. J Hered 86, 248-249.
Ryan A M, Womack J E (1993) Somatic cell mapping of the bovine prion protein gene and restriction fragment length polymorphism studies in cattle and sheep. Anim Genet 24, 23-26.
Sander P, Hamann H, Drogemuller C, Kashkevich K, Schiebel K, Leeb T (2005) Bovine prion protein gene (PRNP) promoter polymorphism modulates PRNP expression and may be responsible for differences in bovine spongiform encephalopathy susceptibility. J Biol Chem 280, 37408-37414.
Sander P, Hamann H, Pfeiffer I, Wemheuer W, Brenig B, Groschup M H, Ziegler U, Distl O, Leeb T (2004) Analysis of sequence variability of the bovine prion protein gene (PRNP) in German cattle breeds. Neurogenetics 5, 19-25.
Schreuder B E, van Keulen L J, Smits M A, Langeveld J P, Stegeman J A (1997) Control of scrapie eventually possible? Vet Q 19, 105-13.
Scott M R, Will R, Ironside J, Nguyen H O, Tremblay P, DeArmond S J, Prusiner S B (1999) Compelling transgenetic evidence for transmission of bovine spongiform encephalopathy prions to humans. Proc Natl Acad Sci USA 96, 15137-15142.
Vilotte J L, Soulier S, Essalmani R, Stinnakre M G, Vaiman D, et al. (2001) Markedly increased susceptibility to natural sheep scrapie of transgenic mice expressing ovine PrP. J Virol 75, 5977-5984.

That claimed is:

1. A multiplexed PCR system for identifying a population of cattle or an individual therefrom that possesses an elevated level of putative resistance or susceptibility to bovine spongiform encephalopathy (BSE), comprising a mixture of primers targeting bovine prion protein gene (PRNP) insertion/deletion polymorphisms in the putative promoter, intron 1, and 3′ untranslated region of the bovine PRNP gene, wherein one or more targeted PRNP insertion/deletion polymorphisms are associated with BSE.

2. The system of claim 1, wherein said one or more PRNP insertion/deletion polymorphisms comprise the 23 bp insertion/deletion in the putative promoter, 12 bp insertion/deletion in intron 1, or 14 bp insertion/deletion in the 3′ untranslated region of the bovine PRNP gene.

3. The system of claim 1, wherein said system amplifies DNA of the putative promoter, intron 1, and 3′ untranslated region of the bovine PRNP gene simultaneously in a single reaction.

4. The system of claim 1, wherein the primers are labeled.

5. The system of claim 3, wherein the primers are fluorescently labeled.

6. A method for identifying an individual cow, bull or calf that possesses an elevated level of putative resistance or susceptibility to bovine spongiform encephalopathy (BSE), comprising the steps of:
(a) obtaining a DNA sample from the cow, bull or calf; and
(b) amplifying the DNA sample using a mixture of primers targeting bovine prion protein gene (PRNP) insertion/deletion polymorphisms in the putative promoter, intron 1, and 3′ untranslated region of the bovine PRNP gene, wherein one or more targeted PRNP insertion/deletion polymorphisms are associated with BSE,
wherein the presence of amplified DNA product of the bovine PRNP putative promoter, intron 1, and 3′ untranslated region corresponding to said one or more PRNP insertion polymorphisms indicates that said cow, bull or calf possesses an elevated level of putative resistance to BSE, or wherein the presence of amplified DNA product of the bovine PRNP putative promoter, intron 1, and 3′ untranslated region corresponding to said one or more PRNP deletion polymorphisms indicates that said cow, bull or calf possesses an elevated level of putative susceptibility to BSE.

7. The method of claim 6, wherein said one or more PRNP insertion/deletion polymorphisms comprise the 23 bp insertion/deletion in the putative promoter, 12 bp insertion/deletion in intron 1, or 14 bp insertion/deletion in the 3′ untranslated region of the bovine PRNP gene.

8. The method of claim 7, wherein said one or more PRNP insertion/deletion polymorphisms comprise the 23 bp insertion/deletion in the putative promoter or 12 bp insertion/deletion in intron 1.

9. The method of claim 6, wherein the primers are labeled.

10. The method of claim 9, wherein the primers are fluorescently labeled.

11. A method for identifying a population of cattle that possesses an elevated level of putative resistance or susceptibility to bovine spongiform encephalopathy (BSE) collectively, comprising the steps of:
(a) obtaining DNA samples from the population of cattle;
(b) amplifying each of the DNA samples using a mixture of primers targeting bovine prion protein gene (PRNP) insertion/deletion polymorphisms in the putative promoter, intron 1, and 3′ untranslated region of the bovine PRNP gene, wherein one or more targeted PRNP insertion/deletion polymorphisms are associated with BSE; and
(c) detecting amplified DNA product of the bovine PRNP putative promoter, intron 1, and 3′ untranslated region corresponding to said one or more PRNP insertion/deletion polymorphisms; and
(d) determining the frequency of said one or more PRNP insertion/deletion polymorphisms,
wherein a significantly higher frequency of said one or more PRNP insertion polymorphisms in said DNA samples than that observed in a reference cattle population affected by BSE indicates that said population of cattle possesses an elevated level of putative resistance to BSE, or wherein no significant differences between the frequency of said one or more PRNP deletion polymorphisms in said DNA samples and that observed in a reference cattle population affected by BSE indicates that said population of cattle possesses an elevated level of putative susceptibility to BSE.

12. The method of claim 11, wherein said one or more PRNP insertion/deletion polymorphisms comprise the 23 bp insertion/deletion in the putative promoter, 12 bp insertion/deletion in intron 1, or 14 bp insertion/deletion in the 3′ untranslated region of the bovine PRNP gene.

13. The method of claim 12, wherein said one or more PRNP insertion/deletion polymorphisms comprise the 23 bp insertion/deletion in the putative promoter or 12 bp insertion/deletion in intron 1.

14. The method of claim 1 1, wherein the primers are labeled.

15. The method of claim 14, wherein the primers are fluorescently labeled.

16. A kit for identifying a population of cattle or an individual therefrom that possesses an elevated level of putative resistance or susceptibility to bovine spongiform encephalopathy (BSE), comprising, in a suitable container, a mixture of PCR primers targeting bovine prion protein gene (PRNP) insertion/deletion polymorphisms in the putative promoter, intron 1, and 3′ untranslated region of the bovine PRNP gene, wherein one or more targeted PRNP insertion/deletion polymorphisms are associated with BSE.

17. The kit of claim 16, wherein said one or more PRNP insertion/deletion polymorphisms comprise the 23 bp insertion/deletion in the putative promoter, 12 bp insertion/deletion in intron 1, or 14 bp insertion/deletion in the 3′ untranslated region of the bovine PRNP gene.

18. The kit of claim 17, wherein said one or more PRNP insertion/deletion polymorphisms comprise the 23 bp insertion/deletion in the putative promoter or 12 bp insertion/deletion in intron 1.

19. The kit of claim 16, wherein the primers are labeled.

20. The kit of claim 19, wherein the primers are fluorescently labeled.


SEQUENCE LISTING (10 sequences)

SEQ ID NO: 1 - DNA, 78056 bp, Bos taurus
taggaataatcaatattgtgaaatgaccatataccaaatgcaacctacagattcaatgagatctccatctaacttccaatagcatttttcacagaagtagaacaaaaaatttcacaattcatatggaaacacaaaaggccctgaatagccaatgcagtcctgagaaagaagaatggagttggaggattcaaccatcctgactttagattatactacaaagctacagtcatcaagacagtatggtattggcataaaaacagaaatatagacaaatggaacaagacagaaagcccagaaataagcccatgaacctatgggtaccttattcctgacaaaggaagcaagaatatacaatggggcagacagcctcttcaataaatggtgctgggaaaactggacagctacatgtaaaagaatgaaattagaacacttcctaacaccaacagttcagttcagttcagctggtcagtcgtatcgactctttgcaaccccatggactgcagcatgccaggcttcccttgtccatcaccaactcctagagcttactcaaactcatgtccattgagttggtgatgccatccaaccatctcatcctctgtcgtccccttctcctcccaccttcaatcattctcagcatcagggttttttccaatgaggcagttctttgcatcaggtggccaaagtattggactttcagcttcagcattagtccttccgatgaatattcaggactgatttcctttaggatggactggtttgatcttgcagtccaaatgactctcaagagtgttctccaacaccacagttcaaaagcatcaattcttcagcactcagctttctttatagtccaactctcacaaccatacatgactactggaaaaaccatagctttgactagatggagctttgttggcaaagtaatgtctctgctttttaatatgctgtctaggttggtcataacttttcttccaaggagcaagcatctttaatttcatggctgcagtcaccatatgcagtgattttggagcccccaaaataaagtctgtcactgtttccactgtttccccatctatttgccatgaagtgatgggaacagatgccatgatcttagtttcctgaatgttgagttttaagtcaactttttcactctcctctttcactttcatcaaaaggctctttaggtcttcttctcttaaccataaggatggtgtcatctgcatatctgaggttattgatatttctcctggcaaacttaattccagcttgtgctttatccagtccagcatttctcgtgatgtactctgcatataaattaaataagcagggtgacaatatacagcctcaatgtactcctttcctgatttggaaccagtatgttgttccatgtctagttctaactgttgcttcctaacttgcatacagatttctcaggaggcaggtcaggcgttctggtattcccatctctttaagaatttcccacagtttgttgtgatccacacagtcaaaggctttggcacagtcaataaagcaaaaatggatgtttttctggaacgctcttactttttcgatgatccaatggatgttggcaatttgatctctgattcctgtgccttttctaaatccagcttgaacatctggaagttcatggttcatgtacttttgaagtctggcttggagaatttgagcattactttgctagtgtgtgagatgagtgtaatcatgcagtagtttgagcattctttggcattgcctttctttgggattggaatgaaaactgaccttttccagtcctgtggccactgctgagttttctaaatttgctgccatattgagtgcatcactttcacagcatcatcttttaggatgtgaaatagctcaactggaattccatcacctcccctagctttgttcataatgatgcttcctaaggcccacttgacttcacattctaggatgtctggttctaggtgagtgatcacaccatcatggttatctgggtcatgaagttctttcttgtagagttcttctgtgtattcttgccacctcttcttaatatcttctgcttttgttaggtccataccatttctgtcctttattgtgcccatctttgcatgaaatgttcccttggtatctgtaattttcttgaagagatctctagttcttcccattctattgtctccagggtgaccacccccagaccctgtacctggggtcatataaccacagtttccacagtgacatcccttccaaaccctgaacttggggttgcacgtccatgttctccagcgtgacacaccctcttggaccatgcactggggtcacatgtccaggttccaggctaacacccccccccatactctatacctggcatcaacgtcctcatacagcagggtgaccgaaccctcaacatcatgtacctggtgttgaaactccacagtttctgcacctcatcagaccttgtacctggagtcacatgtccacagtctctagggtgacagcacttcaccagacctttgaccagtgttcacacatccagtctccagggtgatgcccacttccagaccctgtacctggggtacatgttcacagtctccagggtgacagccccccagactctgtagctgggttaacagacccacctttccagggtgactacaccactccatattgtgcacctggggtcacacatccacaatctctggggtgacctctcccagaccctgtacgtgggtcacacatccacagtcccagggtgaccccacttcccagactgtggaactgggttcacatgtccacagtttccaggtgaatccccctccccaaagcctgtacctggggtcacacatccacagtctccagagtgaccctagcctccagaccctctccctggggtcacatgtccatggtctacagggagatacccctcccagaacctgcacctggggtcacatggccacagtctccagggtgaacccctaccagaccctgtacctggggtcacatgttcagagtctccagggttacctgcctcccagaccctgcaccttggttcacatgtctgcagtctccagtgtgaccccactcctgtacctgtggtcacatgtgcagattccagggtgacacccctcccagaccctgcacctggggtcacacgtatgccatctccagggtgaccccgcctcaccagaccttttacctggggtcacaccttcacagtctccagggtaacccccccacccagactctgcacttggggtacaaatccacagtttccagggtgacaccccctcagaccttctacctgaattcagagtttgatagcctcctgggagaccccaccacaccagcgagtgcacctggcttcacacgtccacagcgtccaggatgacatgcccccagaccctgtacctagggtcacatatctctagttccctggtgacccctccaagaccctgaacctggggtcatatgtctgcagtctccagggtgaccaaccacagacactctacctggggtaatatattcacagtctacagggtgacaacccactcagaccctgaacctggggacacatgtccacggtctccagggtgatcacacactccagaccctgtacctggggtcacatatccacagtctccagggaaacccaactgcccatactgtgcacctgggggtcacacatccagtctccagggtgaccccccgccccatatcctgtaccttgggccacatgacctcagcctccagggtgaccccaccctcaacatcatttacctggggccaaatctccacactctccagggtgacctcctcccagaccctgcacgtggggtcacatgtccacagtctccagggtgaccccatgtcacagatcctgcacctgagtcacatgtcaaccgtctccatggtgacccctcccagactgcacctggggtcacatatccatagttcccatggtgatcccaccctggccctgtacctgtggtcacatgtccacagtttcagggtgagttccctcccacgttctgtacctgaggtcacatgtccatagtctccagggtgaccccatcttctagacattgtaccagggttcacagatccacagtctccagggtgatctccctctccataccctgtacctggggtcaacatcctcagcttccaggatgacccaatcctcaacatcgtgtaccgtgggtcaaacgtccacagtctccagggtcactgcacctcactagaccttgtacctggggtcacatgtgcacagtctctagggtgacattacctcaacataccttttaaatgggttcacacgtccacagtctcccagggtgactcccctctcagcctcctgcacctgagatacacattcacagtctccagggtgacatcccccccagacactgtacctgggttcacaggtccacctcctccagggtgacctcaccacaccagaccacgcacctgtgatcacacatccacagtgtccagggtgacaccctcccagatcctgtacctaaggtcacatatctacagttccctgagagaccctcccaaaccctgtacctgggtcacacatccacagtcccagggtgaccccacttcccagactgtgaaaccgggttcacatatcaatagtttccaggtgaattcccctccccaaaccctgtacctagggtcacacgtccacagtcccagggtgaccctagcctctacctggggtcacatgtccacattctacagggtgacccccctcccaggccctgctcctagggtcatatggccagtttccacagtaaacccttcccagaccctgtacctggggtcacatgtccagagtctccagggtgatccacatcccaaactcttcacctggcatcacacgtccatagtctcaagggtgacaccctcccagactctgaacctggggtcacatgtccacagtctccagggtgacccccacccgaccctgcccctggggtcacacgtcttcagtctccagggtgacacccctccccagacactgtaactagagccacatgtccacagtctacaagggtgaaccccgccccccccccataatctgcatgtgggttcacatatccacagtctccttggtaaccttgcttcccagtaacggcacctggattgcacatccacagtcttcatggtgaccccctcccagactctgcacctgagttcaaatgtctacagtctccagggtgacccctcccaaaccctgcagagggcctcacatgtccacagtctccaggctgaacccccctcccagaatctatacctggagtcacatgtccacagtctccagggtgacaccccctcccccagaccttgggggtcacattgaaacagtctccaggtgaccatcttcccagaccctgcacctagggtcacaagcccacatctccaatgtgacccctctcctgctccaggggtcacatgtccacaaattccaaggtgacacctctcccagacactgcacctgggttcacatgtcccactgtctccagggtgacaacccccatactctgtacctgggttcacaggtccacagtctctagggtgactctgccacatcagactgcacagctgtgggcacatgtccacagtttccagggtggcaccctcccagatcctgtacccagggtcacatatttacagtcccctgggttaacactcccagaccctgtacctgaggtcatttgtccacgttgtccagggtcaacccttcccagaccctacacctcggctcacatgtccacagtctccagggtgacctcctcccaaccctgaacttggagtcacatgtccagtctctagggtgatcacccccataccctgtacctgggatcacaaaactacagtctccagggtgaccctgtgcccagacactgaatctggggtcacacatccacagtctccagtgtgatgcccactcccagaccatgcaccaggggtcacacatccacagtctccagggtgacagcctctgagaccctgaacctggggtcacatgcccacagtctacagggtgacgacccctcccagaccacatacatgggttcacaggttcacagtctccaggataacaccctcccagaccctgtacctatggacacctatctacaatccccttggtgtcccctcccagacactatacctgggttcacatgtccacagtcttcagggtgacaccctccagaccctgtacctagggacacctatctacagtccccttggtgtcccctcccagacactatatctggattcaaatgtccacagtctccaggttgaccccaccctcaagaacctctacctgtagtcatatgaccacagtctccagtgtaaacccacctcccatatcctgcacctggagtacatgtccagtctccagggtgaccccacagtccagaccctgtacatagggtcacagacctcagcctcaagagtgatactctcccagagtttttacctggggccacatgttcatagtctccagggtgatcctctctcaactctgcacctggggtcacacatacaaagtctccaggtaacaccccccaataccctgtgcctggtgttgcacatccacagatttcacagtgaccccacctcccggaccctgcatctgaagtcaaatgcccacagtctccagtgtaaactcacatcccatgctgttcctggggtaaatgtccacattatccaggatgaccccacctcccagaccctgttccttgggtctcacttacacagtctccagagtgtccccacctctcagaccctccatctgggatcgaacattgccagtatccagggtgaccccctcccagactctacacttggggtcacatgtccagagtttccagggtgactgcctcccgaacctgtagctgtggtcacaggtccacagtcttcagggtgacacccctctccagacaatgtatctggagtcacacattcacagtctgctaggcgaacccagcccccaaaccctgcacatgggaccccatgttcagtggggagtgttgactaccagcctacaacaaccagctggctgttgctgtcagcagcctaattgggtgaaaaatggactgggtgatgacttcccaataagaagtggcaagtagcatgttcctttcagaaaactcaaataatgaaccaaagttgttgtcataatgtacagacaaatgacctggtgtgtttcttcatggttggttgagtctgcaactatccacctttcccaggatgatcatatagattttgccatattactttgattccagcctcaacataacatgtttccctgtacatttagagctgggtaaagacactcctggagaaggcaatggcaccccactccagtactcttgcctggaaaatcccatggatggaggagcctggtaggctgtagtccatggggtcacgaagagtcagacacaattgagtgactttactttcacttttcactttcatgcattggagaaggaaatggcaacccactccagtgttcttgcctggagagtcccagggacgggggagcctggtgagctgccatctatggggtcgcagagtcggacacgactgaagcaacttagcagcagccgcagcaaagacactcctagtgtacaaacactgtacagtttgaggagtatagacagcagtggagagtgctctatgaatgtggatggccaggtctgtttttaccctgagtaggtgaaacgtactgtcaggtgacctcacagcaagaagtggcaagctgcctgtcatgagaagtgaagttgcccactcatatgcgactctttgcaatcccatggactgtagcctatggaggtcttctgtccatggaattttccagtcaagagtactaaagtgggttgccaattctttttccagggaatcttcgcgacctgagtatcaaacctgggtctcccacatcgcaggcagacactaccctcttagctaccacggaagcccaccagattcattgaaaactcaactgtttacccgaagttgttctgggattgaagagtagaatgagcctgctgtgtttttctgccaggtgggatgctgtaactacacaggtttcacagggggaaatgactgctttggaacgtccgatcccaattccaggttcacaaaaagttgtctccctgcagttttaaagctgtgtaaatgccttcctaggacacaatgggctctctgtgaatgttgacttctgtatgtgttcttaccctattcagcgaaaacagtactgtcagaggacttcccatcaagaagtggcaagcagagtgtttctctcaggtaacagaagtacttgatgagacttgcagtcctaaagaagggaaaaacgacccgtgtgcttttccgtcaggctggagattgcaagtattccaggtttgctaggtgaaaatactgctttctcttcaggccccgagttctacgttcagagaaacatttccacacaggttcagagctgtgccaagattccaagaacacactgtacaatttctcagaaaagacttcagtgaagaatgtcctgcaagtggttctgccataattgttaccctgagtgggtggaattacactgtcagatgactttttagcattaaaaagcaagtgcctatgctttcagaaaaggcagatactaaagctgcgctcgtaagaaacaggcaaatgaacccgtgtgcatttctttcaggcttaagactgcaaccaccctgcagttacacgaaaaagtgctgcttctgccattaggctccaaatcccatgtgaacagaaataaatccccctgtcattagaaagctgtgtaagtgagcccaggccagaatgcaccataccatgagtaaagaggtcagtggggagtgttctgagagttcaatagtgagtgttctggaagtgttgactgccatacctgtagtcagcagcctaatttggtgaaactggactgggtgatgactttccagtaagaaggagcaagctgcaattacctttcagaagactcataatggcccaaagctgttgtaataatgcccagacaaatgagtcactgcgtttcttcctagctggttggtggctgcaactatccaggtttaaggggaggaatatactgactttgccatattaccttatttgcagcttcaggtaacatatttccctgcacatttagagctgtgtaaagacactcctagagaacaaactgtaccatttgtggagcaaagatagcggtggagagtgctctgtgaatgtggatggtcaggcctgtttttagcctgatgaggtgagatgtaatatcaggtgacctcacagccagagtggcaagccacctgattcatgagaagtgaagttgctcagttgtatgtgactctttgcactcccatgcaatgtagcttaccaggctcctttttcgatggaattttccaggcaagaagtactggtatggcttgccacttccttttccaggagatattcctgacccagggatcacacccgggtcttcaatgttgcaggcagatgctttaccctctgggtcaccagaatccggctggattcattgaaaattcactgatttaacaaaagctgtcctggaatggaagagtagaatgagcctgttgtatttctctggcaggtggtatgctgtaactacaaggcttcacaaggacgaatgctttgggacatcagtccccaattccacgtgcacaaaatgacatctacctatagtttcaaagctgtgtaaatccattcctaggacacaatgggctatccgtgaaggttgacttttgcatctgtttttacactgatcagctaaaactgtactgtcagtggacttcccatcaggaagtggtaagcagaatgtttctctcagaaaacacaagtccttgatgagacttgcggccctaaagaagggaaaatgcccccgtgtgttttctgtcagtctggagactgcaattattccaggttccttaggcacagattttgctttcatttcaggccctgagttctaggttcagagaaacatttccccacagcttcagagctgtgcaaaacactcctagaacacactgtatcattcccttagacaagagaccagggaagagcattctgtgagcgcttctgcgatatcagttgttaccctgagtgggaagaattacactgtcaggtgacttggtagcattaataagctagagtctgtgctttcagaaaaagcagacacttttagcaaagttgccttcgtaaggaagtggaaaatgaacccgtgtgcctttctttcaggcttaagactgcaatcaccctgtggtcaaaaaagaaatactgcttctgacattaggctaaaaagcccacatgaaccaaacaaatgcccctgtcgttagaaagctgtgtaagagacccaaggacagactgcaccagtccatgagtaaagagttctggggggagtgttctctgggtgttgactgccatacctgtagtcagcagcctaattcggtgaaactggactgggtgatgacttcccagtaagaagtggcaagctacatgttccttttggaagactcaaagaatggccaaaagctgttgtcataatgcccagacaaacgagccagtgcatttcttgctggttgaaggctgcaactatccagctttcagcagaggaatattctgattttgccatattacctggatttgcagcttcaagataacacatttccctgcacaagtagaggtgtgtaaagacactcctagtgtacaaactctgtaccacctggggaacaaagatagcgtggacagtgctctgtgaatgtggatggccaggcctgttttcaccctgaagaggtgaaaagtactgtcgggtgaccttgcagccagaagcggcaagacgcctgcttcatgagaagtaaagttgttcagtcattgcgactctttacaatcccgtggactgtagcttaccaggctcctccatccatggaattttccaagcaagactactggagtggcttgccatttaattctcaatggatcttcctgatccaaggatcaaacccaggtcacccatgttgcaggcagatgctttaccctttgagccaccagggaagcctgccagattcattgaaaattcaactacttaactaaagctgttctggaatggaacagcagaatgagcctgttgtgtttttctggcaggtgagctgctgcaaccacacagtttcccaaggggaaatgactgttttgggatgtcagaccccatttccaggtgcacaacaggacgtctccctgaagattcaaagctgtgtaaacgcattcctaggatacaatgtgctctctgtgaatgttgacttttgtatctgtttttctactgattagctaaaactgtactgccaagggacttcgcatcaaaaagtggcaagcagacccatggtcctaaagaagagaaaaatgaccccatgaacttttccatcaggctggagattgtaagtattccaagttcattagtaacaaatgctgctttcatttcaggccaccatttctaggttcagagaaacatttccccgcagattcagagctgtgcaaagacactcctagaacacactgtatcattccctaagaaaagagttcagggaagagtgttctgtgagtacttctgccatacctgttgttaccctgagtgggctgaattacagtgtcaagtgactacgtagcattaaaaagcaagggcgtgttctttttgaaaacacagatacgtaagtaaaactaccatcataaggaagaggcaaatgaatgcaagtgcatctctttcaggcttaggactgaaaccacgctgcagtcacaccaagtgctgcttctgccattagtctctgaatcctacgtgagcataaaaaaaaaaaaaaaaaaatccctgttgtgagaaagctgtgtaagagaccccaaggacagattgcatcattccatgggtaaagagttcagtgaggagtgttctgggactgttgactgccatacctgttgtgagcagcctaattttggtgaaactggaccagttgatgacttcccagtaagaagcggcaagctgcacattccttttggaagactcaaagaatggccgaaagctgttgtcataatgcccagacaaatgagccattgtgtttcttcttggctgtttggaggctgcaactatacatctttcacaggaggaatatactgactttgccatattacccagatttgcagcttcaagataacacatttccctgcacatttattgctgtgtaaagacacttctagtgtacaaattctgtgccgtttgtggagcaaagatagcagtggagagtgctctgtgaatgtggatggccaggcctgttttcaccctgatgaggtgaaaaatactgtcagatgaccttgaaccaagaagtggcaagccgcccgcttcatgagaagtgaagtttctcagtcatgtttgactctttgcaatcccatggactgtaccttaacaggctcctcagtccacggaattttccaggtaacagtactagagtggcttgccatttccttctccagggcatcttcctgacccaggttttgaatgcgggtgttccactttgcaggcagccgttttaccctctgagccaccaggaaagcctgctggggtttacagaaaattcaacttcttaaccaaagctgttctggaatgaaaaagtagaatgagcctgctgtgtttttctggcaggtgggatgctgaaaccacacaggttacccaaggggaaatgactgctttgggacatcagacaccaattccaggtgcacaaaaagatgtctccttgcagagtcaaagctatgtaaatgcattcctaggacacaacgtgttctccgtgaatgttgacttttgcatccctttttgcactgatcctctaaaactgtactgtcagaggaattcccatcaagaagtggcaagcagacttgtagtgctaaagaaggggaaaatgcccccgagagctttctgtcaggctggagattacaagtattccaagttcattagtaacatatgctgctttcatttcagggcctgagttctaaggttcagagaaacatttccccatagcttcagagctgtgcaaagcgactcctatcacacactgtatcattgccttagaaaagagttagggaagaatgctatgtgagtgcctctggcatacctgctgtcaccctcatgggctgaattacactgtcaactgactatgtagcattaatcagcaagtgcctgttctttcaccaaacgcagatatataagctaagctgcggtcatgaggaaaaggcaaatgaacccatgagtgtttctttcagggttaaaagtgcaagcaccctgcagtcacacgaagaaatgctgtgtctgcctttaggcaaaaaatcacaggtgaacataaacaaatatccctgtccttagaaagcagtgtaagacagcccagggacatattgcaccattccatgagtaaagagttcagtgaggagtgttctgggatacctgttgtcagtagcctagtcggtgaaactggactgggtgacgacttcccagtaagaagtggcaagccgcaggttccttttggaagactcaaagaatttcccaaagctgttttcataatgcccaggcaaatgagccagtgcgtttcttcatggctgattgcaggctgcaactatccagctttcacagtagaaatatactgattttgccatataacccagatttccagcttcaagagaacacatttccctgctcatttagtgctatgtaaagacacccctagtgtataaattctgtaccatttgcagaggaaagagcagtggagaatgctctgtggatggccaggcctgtttttaccctgaagaggtgaaaagtactgtcaggtgacctcgaatcaagaagtggcaagcctcccgcttcatgagaagtgaaggtgctcagtcgtgttggctttatgcaatcccatggactgtagctgaccaggctcctctgtccatggaatcttctaagcaagaagactggagtgacttgtcatttctttctccagggaatcttcccgacacaggatcgatccctggtctccctcattgcaggcagacgctttaccctctgagccaccagaggaacccgccagattcactgaaaagtcaaccacttactgaaagctgctctagaatggaagagcagaatgagcctgctgtgtatttctggtaggtgggatgctgcaactacaaaggtttaccaggggaaatgactgctttgggacattagtctccatttccaggtggacaggacgatgtctctgcacttttcaaagctgcgtaaatgcattcctaggacacaatgtgctctccaagaatgttgacttttgcatctgtttttgcactgatcagctaaatctgtaccgtcagaggacttcccatcaagaagtgacaagcagactttggtcttaaagtagggaaaaatgcccctgtgagcttttccggcaggatggagattgcaagtattccaggttctttaggtgcaaatgctgctttcatttcaggccccaagttctaggttcagagaaacggtttgcccagcttcagagctgtgcaaagacactcctagaacacactgtatcattcacttagaaaagagtataaggaatagtgttctgtgattcatctgccatacctattgttaccctgagtgggccaaaatacactatcaggtgactttgtaacattaacaggcaagcacctgtgctttcagaaaatgcacatacttaagcaaagctgtggtcgtaaagaagaggcaatgaacccatggatgtttctttcagggttaaggttgtaaccaccctgcagtgacacaaagaagtgctgcttctgccattaggctaaaaatccctggtgaaccaaacaaaagtccccctgtcattagacagctatgcgactgagtccaaggacagattgcaccattccatgtgtaaagagttcagtggggagtgttctgggactgttgactgccatacatgttgtcagcagcctaattcagtgaaactggactggatgatgacttcccaataggaagtggcaagctgcatgttcctttcagaagactcaaataatggtacaaagctgttgtcataatgctcaaatgagacagtgcatttcttccttgctggttagaggctacaatccaggtttcacaggaggtatatactgattttggcatattacccggatgtgcagcttcaagataatacatttcccagcatttagagctgtgtaagacaccctagtatacaaactctgtaccatttgtggagcaaagacggtagtggagagtgctctgtgcatgtggatggccaggcctgtttttaccctgatgaggtggaaagtactgttgggtgaccttgcagccagaagtggcaagctgcctgcttcatgagaagtgaaattactcagtcgtgtggctttctgcaatcccatggactgcaccttaccagactcctccatccatggaattctccatgcaagagtactggaatggcttgccatttccttctccagagggtcttcctgacccagggatagaacccaagtcccccatgtggcaggcagacactttaccctctgagccactagggaagtgtgccagattcagtgaaaactcaactacttaaccaaagatgttctggaatggaagagcagaatgagcctgttgtgtttttctggcaggtgggatgctgcaaccacacaggtttccccaaggggaaatgactactttgggacttgagaccccaattccagttgcaccagaagacatctccctgcagtttcaaagctatgtaaatgcattcctaggacacagtgtactctccatgaatgttgacttttgtatctctttttgcactgataagctaaaactgtactgttagaaactttccatcatgaaagggtaagccaacttctggtcctaaagaaaggaaatgtgcccccgtgagcttttctgttaggctggagaaacaagtactccatgtttgttaggcgcaaatgctgctttcatttcaggccctgagttctaagttcagagaaacatttccccacagcttcaaagctctgcaaagacactcctagaacaaacagtatcattcacttaaacaagagaccagggaggagtgttctgtgagtgcttctgccataccagttgtttccttgagtgggcagaattacactgtcacatgacttcgtagcaacaataggcaagaggctgtgctttcagaaaatgcagacacggtctgcattttagcaaagctgtgatcctaaggaagaagcaaatgaacccatgtgcatttctttcaggcttaaggctgcaaacaccctgcagtcacatgaagaagtgctgcttctgccattagactccgaaccccatgggagcattaaaaaatgtccctgctgtgagaaagctgtgtaagagaccccaggacacactgcaccattccatggaaaaagagttcagtgggatatgttctcagagttcagtgtggagtgttctgggactgttaactgccatacctgttgtcagcagcctaatttcataaaattggactgggtgatgacttcccagtaagaagtggaaagctgcacgttcctttcggaagactcaaagaatggccgaaagctgttgtcataacgcccagacaaatgagccagtgcatttattcctggctggttggaggctgcaactattcagctttcgcaggacaaatatactgatttttccattttatctggatttgcaccttcaagataacacattcccctgcacatttagtgctgtgtaaagacactcctaatgtacaaactctgtaccgatgtggagcaaagacagtagtggacagtgctctattaaggtggatggccaacctgtttttaccatgatgaggtggaaagtactatctgatgacctcacagtcagaagtggcaagccgcctgtttcatgagaagtgaagtttctcagtcgtgtgcaactctttgcaatcccatggattgtagctaaccaggctcctctgtccatggaattttccaagtagactagagtggcttgccatttccttctccaggggatcttcctgacccagggatcgaacccgggtctcccacaatgcagacagacactttaccctctgaaccatgggtccattgaaaattcagctacttaaccaaagctgttctggaatggaagagtagaatgagcctgttgtctttttctggcaggtgggatgctgcaactacacaggtttcccaaggggaaatgactgctttgggacatcagatcccaattccaggtgcacaagacatttccctgcagtttcaaagctgtgcaaatgcatcctaggacacaatgtgctctctgtaatgctgacttttttatctgctttttcactgatcagctaaaactgtactatcggaggacttcccatcaagaaatggcaagtagcgtgtttctctcagaaaacataagtacttgatgagacttgtggtcctacagtgggggaaaatgcccccgagtgcttttccatcctgatggtgattccaaatatttcaggtttgatgggcacaaacgctgctttcatttcaggccccgagttctaggttcagagaaacgtttccccacagcttcagatctgtgcaaagacactcttagaacacactgttaccattcccttagaaaacagaccagggaagagtgttctgtgagtgcttctgccataccagttgttatcctgagtgggcagaattacactgtcacgtgactttgtgacattaataagcaagcgcctgtatttcagaaaatgcagacactttagcaaagttgcctttgtaaggaagaggcaaatgaacccatgtgcgtttctttcaggcttaagattgcaaccaccctgcagtacacgaagaaatgctgcttctccctaggctccaaatcccatgtgaaccaaatgtccctgcaaatagggaaaaatcagtatgttcctcctgggaaagcaggagagttgtagcctccagccagccacgaaaacaccccagggcattccattgctcattatgacaacagctttggtcaattatatgagtttgctgagaggaacatgctacttgccactgcttactgggagttatcacccagtccactctcaaagaaataggctaaccacaggtatggcagtcagcaggcacagaaccctccccactgaactctcagaaccctccccactcaactcttaactcttggaatggtgcagtctgtccctgggctctgtgacacagctctctaactacagggacgtttgtcttcttaaaacgggatttggagtctaatagcagaagcagcatttcttcgtgtgactgcagggtgcttgcggtccttagcctgaaagaaacacacatgagttcatttttctcttcctgaagaccggagctttgctaaagtatctgcgttttctgaaaacataggcacttgcatattaatgctacaaagtcattggacagggtcctttggcccactcagggtagcaagaggtatggcagaagggctcacagaacactcttccctgaactcttttctaagggaatggtacagtgtattctaggagtgtctttccccagctctgaagctgtgggaaaaacatcttactgaacctagaactcggagcctgaagagaaagcaaccttttcccctaacaaaacttgaatacttgcagtctccaacctgacagaacaccacactggtgcatttcccccttcttcatgacagcaagtttcatcaagtacttgtgttttctgggagaatcacgcttcttgccttttttttgtgggaaagtcctctgacagtacagttttaactgttcgggataaaaataggcagagaagtcaacattcacggatagtacattgtgtcctaggaacggactcactcagctttgaaactgcagggagacgtgtgttttactacctgggattggcttagggcatcaaacagcagtcatcttctgtgaaacctgtatcattgcagcaaccaacctgccagcaaacccacaggctcattctattcttcagtcctagaacagcgttggttaaggagctgagtttttagtgaatcaggcgggcttcccggtggctcagggggtagaacatctgcctgcaatgcgggacacctgggtttgatccctgggtcgggaagttccacccaccagagaaggaaatggcaacccatttcagtactcttgcctggaagctacagtccatgggaaagagtcgaacacaacttagcagcttcatatctcatgaatcaggctgcttgccacttcttgctgtgagctcatctgacagtacatttcacctaatcagagtaaaacagccttggccatccacattcacagagccctctccatttatatctttgctcagcacatggtacagtgtatgtacactaggagtgttaacatgggatttggagcctaatggcagaagcagcatttctttgtgtgactgcaggtggcttgcaactccttgcatcaaggtcctttccattggaatgtcaactcatctaggtaacaacactatggcactcagcactctcaaatactctcccacagattttcctcatgggcagtcccagtatgaccaaggaatctgttcactagtctcagaaagtggagggaaacatactggctgtgactctcaaaattagggcctgtgtgcaaatgtggtgttctcacatcataaaatctagacagctgctgagagagagatgcctgaaaatcactcttttttcgcctttcatgcatcaaagcactgtacgtttcccagcgttctgtttgctgagagtaacccactacttgtgaatccttgcttggaggtaacaatttcccctcacctgtttcacagtacagaaggaaagcataatcgctgctgcggccgctaagtcgcttcagtcatgtctgactgtgcaaccccatagacggcagcccaccaggctcctctgtccctgggattctccaggcaagaatattggagtgggttgccacttcttctccaaagcataatcacagggagaaacaagacaaaaaccccaagttccaacaacttttctctgtaggaaggatccagtgatcctaggtctgtcttgaaaattcttggatactgcagggatacctactgagtgtgaacctggatattgcaccttgatgacaaaattagcattgtctcttggttgactctagaaggctggggtttagcaactgcctgaaaatctatccaatgttgttttcgtactttgacagcaatagctttggttaaaaacattgggatttctgaagcaagtaggcagcttataagttcttgtgtcaaggtcctttccaccagaatgtcaatgaatctaggtaacaacagtatggcactcagcactctcagtcactctcgcgcagactgtttcttcatgggcagtcccaggatgacctaggaatgtattcaccaggctcagaaagtgcagagaaacataccagctgtgactgtccaaattacagcctttgtgcaaacgtggtcttctcatatcatagaatctagacagttgctaacaggtaggtgcctgaaaaacactccccttttttgcctttcattcatcaaagaaacataggtttcccaacattctgtttgctgagaataacaacactacttgcaaatctgtgcttggaggtcattttaactaacaattacccctcacgtgtgtcacagtagggaaggactgcctacgtacagggaaaaacaaacaagccaaaaaactcaagttccttacattgcctctgtagaaaagatccagtgatcctaggtctgtcagggacacacactgcgtgtgaacctggatactgaaccttgctggcaaaattagcattttctctcttatggccctgaaatggtgcagcctagcaactgcctgaaaatccatacaattttttcccctacttttgacaccaatagctttggtcaaaaattgggatttctgaagcaagcagggggcttgcaactccttgcgtcaaggtcctttcctttgggatatcagctaatctaggtaacagtggtatggcactcagcactctcagatactctcgcacagactgtttcctcagggatgtcacaatatgacctaggcatgtgttcacaaggctcagaaagggcagggaaataaactggctgtgactctcgaaattacatcaggagggatgctgcaactgcacagggttcccagggggaaacgactgctctggggcatcagatcccaattccaggtgcacaagaagacgtctccctgcagtttcaaggctgtgtaaatgcattcctaggacacagtgtgctcttcgtgaatggtgacttttatacgtttttgcactgatcagctaaaactgtactatcagaggacttcccatcaagaagtagcaagcagtgtgtttctctcagaaaacacaagtacttgatgagacttgcggtcctacagcagggaaaattgctcctgtgtgtttttccatcaggctggagattgcaagtattccaggttcattaggcgcaaaatgctgctttcatttcagaccccgagttctaggttcaaagaaacatttccccacagcttcagagctgggcaaagatacccctagaacacactgtatcattctcttaggaaagagaccagggaagagtgttctgtgagttcttctgccatacctgttgttaccctgagtgggcagaattacactgtcgggtgatttgaggcattaataagcaagtgcctgtgttttcagaaaacgcagacactttagcaaagttgccattgtaaggaaaaggcaaatgaacccatgtggttttccttcaggcttaagactgcacccaccctgcagtgacggaagtgctacttctgccattaggctccaaaccccatgtgaacataaacaaatgtccctgtcattataaagctgtgtaagagaactcaaggacagactgcatcatttcatgagtaaagagttcagaagggaatgttctgggagtgttgactgccatacctgttgtcagcagcctaatttggtgaaactggactgggtgatggcttcccagtaagaagtggcaagttgcatgttccttttggaagaattaagtaatgaaccaatgctgttgttttaatgcccagacaaatgagccagtgcgtttcttcctgtctggttggaagctgcaattatccagctttcacaggaggaatatactgattttgccgtattatctggatttgcggcttcaagataacacatttccctgcacatttacagctgtgtaaagacactcctagtgtacacactctaccatttgtggagcaaagacagcagtggagagtgctctgtgaatgtggatggttaggcctgtctttaccatgatgaggtgaaaagtcctgtcaggtgacttcacagccagaagtagcaagcagcctgcttcatgagaattaacgttgctcagtcgtgtgcaactctttgcaatcccatggactgtagcttaccggctcctccatccacggaattttcctggcaagagtactgaagtggctttccatttcctactccaggggatcttcccgacttggggatcaaacctgggtctcccaggttgcaggcagatgttttttcctctgagccaccagggaagccagccagattcattgaaaattcaactacttagccaaagatttctggaatggaagatagactgagcctgttgtcctttttgggcaggtgggatgctggaactacgcaggtttcccatggggaaattacttctttgggacatcagaccccaactccaggtgcacaaaaagatgtctccctgcagtttcaaagttgtgtaaatgcatcctaggacacaatgtgctatccttgaatgttgactttcatatctgtttttgcactgatcagctaaaactgtactgtcagaggacttcccatcaaaaagtggtaagcagcatgtttcttcagaaaacacaagtacttgaggagccttgtggtcctgatgaagcaaaaaaatgtccccgtgtgcttttctatcaggctggagattgcaagtatttcaggttcgctaggtgcaaatgctgctttcatttcagcccccaagttctaggttcagagaattgtttccccacagcttcagagctgtgcaaagacactcctaggacagacacactgtatcatttccttagaagagttcagggaagtgtgttctgtaagtgcttctgccataccctttttttaccctgagtgggcagaattacactgtctggtgacttgttaacattagtaagcaagcaccttgccttcagaaaacacagatacttaagcagaactgtggtcataaggaagaggcaaatgaacccatgtgcatttctttcaggcttaagattgcaaccaccctgcagtgacacgaagtgctgcttctgccattaggctcagaaccctaagtaaacataaacaaatgtccctgtccttagaaagctaatgttggtccagtggtttgtgtaagctttgtataggctgagacttatgctgagtttttgtttgtttttcctctgatgggcaaggctgagtgattgggtttgtacttttgttttgtttgttgtttagatgaggagtcctgcacagggtgctactggtggtctggtgatgccagggcttgtattcaagtggtttcctttgtgtgagttcacactatttgatactctctaggattagttctctggttgtctagggtcttggagttagtgctcccactccaaacgcttagggattgatctctctccaaagaccagcttaggtccaaactaccaagaggaatttcacttgaaatgaaagggcctttactttaccaagaggaatttcacttgaaatgaaagggcctttactttaccaagaggaattcactcgaaatgaaagggacttactgaattccaaaagccagagcacaagaacacatggagatctctacccagaggaaactctaccatgccttggttgcagcagatgttggtcctcttctgctttgatctgtccccgctttctgtggcagctgcccagcagaggccatctgacgtcattttgttcattacctggatgaaggggtgtctccttctcagaggagcctgagacaaacaaagggacagtgcagagccatccccgcacggaagcccccttccatttagaaatgtttctcttaagctatgttaatgaactatgtatttagcctagactctgtgtttcttcacttaggttctgcctaagactcagaactgataatggctcaacaaaccagtatgtttttctcatacaattgttctcctaatctatgttaatgagactatgtatttgcttggaaacctgccttcttcaaaattcatgtcaatcattttatggcctgggatgactcaccttgttccaatgttacctcaaaatgcatgttgtgggtgaggggccctggtgccactctctgagttttgagacatttcctttctttaattagtacccttctgataggtgtataagttaccattaaagtctagcagggggggcactctttctgccccttctaatgtctatgttagaagcttaatctcttttatactttaataaaactttatcacacaaaagcttggagtgatcaagtctcataactggccccagattgaattcttctcctccaaaggccaataatcccatcatctttcatggctcagcaacaacctttcaccgggggagctcatctgggattcttcaggacaaggtaaggacacttggagctctagttctttgttctcctggcaaacacattttctgctgtactttactaactctatggtgtgcttgtgtgtgtgattgaaagatgcacacatgtgtgaagcaagatctgggtccaaatctttggttctgtggtgacctcataccacttatggcaggaaccctgttgggggattatactgacctgctaatgtcaagaggcacccaatgtctcctccagggaaacagaccaaggtggataaaacgtgtggatggaactctccttttttggccaaactttctggtctctttgaccatttcataacttcctgggaattagaactactaacctaatcagtgggatcatagactttcaagggacttgttatctatgctgttactgtgtattgtcacttaggttccaaactttgttttgtttttttttgtattcacaaattgcctagcctcactaggagtcaatagtttggaagctagatggagttctaattccaagaacatctctcaggtttaagattactcaggaaatacagcagtcttcttcctttggtaacactagctcttagtggaccagaggaggatctccagttgcttctgtctcaacaccttagatattcttcctgtggaatctgtgggaatgaactggaaggactggccataataacttgaggacaaaaatcactttttcctcagtggccagcccctcaccgtctcttttgctatcgcttatattggtgtggtgagactcagaaggaacatcttggttttatgtttgtcctttatgatttactggtcttactgtggtcaggaatgtactcagggttgtgcataggcactcaggagacaaatatttcccttagtggtcttagcttgggaggcattctggaaggttactctgactgcacctcgggtggcatcagaggcaagcaaaagttttaatggtgaggaactgggtattagtctgggatgccatcaggtctacccctgatgcatctccaccccaccgcagtggtagaatggggaggggcagtagtggaatacctgtggtaagagacaggttaactccagccagggaaggaagcttaggtggaagacctgtctccacccccatctagaacagggagggacagtagaaggacagtgctggtagctgtttttctctcttaaaggtgggagctaaccattccagcctcactcctttgaaaaactgggatagatttgatccccagagcctaatgaagacatgcctgatcttcctatgtgatactacatggccacagtatccattggaggatagcgaatggtggctggttggagggtctcttaattacaatattgttttacaattaaactggttctgtagataacaaggaaatgggtagaagtagcatatgtgttgccctttttctctctgtgagacatatcagatttatgtcctaagggtatatattagggtatgaaattttcagctccctattctgctatatgaccttatttgggaggatgtgatgtatatcctgggacaggcgctaactcctgcatcaacaacttgagtttggaaagctgttgcctatggagatgaatggcttggcaaggaatcattagggaagagggaggatgagatagctgccctccccactggggatcaggcagtcccaactatagaaccagattgggactaaaaggctaaaggatgatgggataagagtcatttgtcagatgtgttcttgaaggactcagacaagctcatgctaagactttaaatgatgctaatttggcaaacatagaacaggaagagaacgaagcttctggtaaattcctagatagactgagggaagccctttgcagattcactgagattgatcctagtcagctccagatatccactaaaacacatgtatggaccaaatcagtctttagataatctgttgcaattggctcagtcagtctattatggcagggagtatgagggaagaaagaaaggcagagaaagaccaaggtactggctgaagcccttgtaatggctgtcaggactgttcttaaacagcctgagaaaaattccaggagagacccaggtgaaaggggatgggcttgctatttctgtggaaaggaggagcgcctcaagcgggattgccctcaggcatctaaggggtccccagctccatgtttgcctgtaaggggccacactggaggagagactgcccccagacgcgtaggtcccagtggtgggattctcaagacaaccaggactgaatgtgcccaggggtccccacacaaactcccaccctaattacagctgaggaaccccaggtattagtaactgtgggtggccaatctgtcaatttccttgtggacaccagggcaagttactctgtgcttactgaagcccctggtccactttctccccaatccgcttctataatgggactgtctggacaagccaaacattaatattttggtcatcctctaagctgtcaactgggactctgtttttacagagtgccagattgtgccagagtctccctcaccccttttagggagggatatactgagcaaggtccatgcctctgtttcatgaatatggagcccttcctttctctccctttaattgaacaaaatgtaaatcctaaagtgtgagctgatggaaaatctgtgggtcgaacacaaaatgctattcctgtagttgtgaagctcaaaaacccactcatacttccccatcaaaagcagtatccactgaaacccgaggttaaagaagggttaaaacccatcatcgagattttaaaggagcaggggctattaattccctataacagtccatgcaacactcctattttgggtataaagaagtcaaattataagtggagactagttcaagatttacaaataataaatgaggctgtacatcctttacaccccatggtgcctaatccttatactctattgtataaaattactgaacaagagaaatatttttcagcatgctttctagagttaaacctatactatgtcatcccctacctatgactttcagacaattgagaggatttgggggaatcataggctactgcctcatttggattctgggttatggggaacttgcctggcctatatatgaacttacaactgaaactcaacaagcccaaactgacaaactggttcagtctctagatactcaaaaggcttttaaagctcttcagattgctctcctgccagctcctgctttaagcttgcccacagggtcagaatttaatttgtttgtcactgaaagaaaaggtatggtcttgggagttttgacacaaccccgagggcctcatcagctatatataggatacctgaaaaacttaaatcctgccactttccttcctgacaaggaaaatgaaacacctgatagcaattgttcccaatttctaactttaaactattcagctcgggaagacctgatggataccccattagacaatcctgatatggaattatttacagatggcagttcttttgttcgggatgggaaacttaaagcaggttacactataatgcgactggacagattttaaaagcaaagtctctccccagggaatgagcgctcaggtagtggagcttgtggctctgacccgagctctagagttaatcaaagggcagcaagtcaatatctacagtgattctaagtatgcttatttgactttacatgttcatgctgtgatacggaaagaaagacagtttaaaacggcaacaggagaacctattaagcatttcaaaagactgagggacttttaactgctataaattgtcctacagaagtagctgttgtgcactgcaaaggacacagtagggatgggaataagtagctgagggtaatcagctggctgactgtcaagccagaaaaccagcagtttaagaaaccccttcactgcagatgcctttgaactagacaggtcctgtggaataggaaaaacatcacaatgaggaagaattagaaagatatgagaaagtaggagcaaacattatcgataaaggatgcttatagtccaaggatggatgatgaataattactgaaaattctcaatggaaaattcttaagagtttacaccagagttttcatttaggtgttgagagcacttaccagatggcttctcatttctttgaaggaaaaatgtaatggaaactttagagaacattatcaaaaactgtgagatttgtcagaaaaataacccaaagactgaaaagttagcaaaatctgggttacaatgaagtggaaaatatcctggagaggactgggaaattgattttactcatatgccaaaggcaaatggatattcttgattacaagtttgggtggatatatttactggacagattgaggcttttccctgtcatagtgaacagcctaagcaggttataagaattttaatccatgaaattactcccaggcttgggctgctgtggagccttcagagtgacaatggctttgcctttaaagccactgtaactcaggggatgtcaaaagctctaggaatagacgatcacttacacggctcctggagaccccgatcctcaggaaaggttgaaaaagctaatgacattattaagagacatctgtgcaaattaactcaagagaggcatgacagttggtgtaaagttctacacatagctttaatgagggctcgaattgccccccaaaatgagggactgtccccctttgagtgcatttatggaagacccttcttacccacagacattgttatagaccttgaagccttggaattatctaactgtgtaactcagcactcagcttttcaacaggcattaaaggaactctgatgtgactcatgacccagactctaagtcaagaaagacactgtctgagccaggaactgaggtcctgataaaaatattgggatctcgggggcaatccctggagcccctctgggaaggcctttaccaggttattctatcttttcccatagctgtcaaagtgccagatattgatcattttacaccacacttaagagttggcatcctgaccagaactaaatgatgtcactttatgtctttattctctacactcttactttgtacttttcagatcagcctgataatctatgtgagcttgcttctgctgactccaaaaatccagtgtctgccgtttgaccctcaagacaatgccctcctgtcctgggatcactcctatgctgcatttcacattcagtctaattactgggtctgtggagcactcccttcttcatcagtggaaggcttccgtggtgggcatctccacttgaaggaaaggagtttcttcaagtctgcaaatctttctacaaagacaataatatgtgatgcctcttcttaatatgataacatctaacaatcctaagatggactggtgcaacactttgtaccttaactatgggcactatgagacttttaactttgctgattgttctgtttttgctgtttgctccctgcatctgtaagagtgtggctggatttgtttctagctgcatgaaggattttaagtgacaaatggttgctcaaactcctgccactgtggcagcttcctccaactacctacttggggcccctggatcagagaccctcaatatgagggttaggagagtatgttgcctcaccaatttagggacgatgccccttatcagcttggaagcagttacagaatgaaaacaatgcccctttccctaggaaacataattctcctaaaagaaaagggagaaataagacggtaacaggcaggaaggctcagttcagttcagttcagtcactcagtcgtgtctgactctttgcgaccccatgaattgcagcatgccaggcctccctgtccaccaccaactcccagagttcactcaaactcatgttcttcgagtcggtgatgccatccagccatctcatcctctgtcgtccccttctcctcctgcccccaatcccttccagcatcagagtcttttccaatgagtcaacccttcacgtgaggtggccaaactattggagtttcaacttcagcatcagtccttccaatgaacacccaggaccaatctcctttagaatggactggttggatctccttgcagtccacaggactctcaagagtcataggttgcaaatgtcagacatttttcatctctctctcaagtggcaggaggaaacaaactgcaagtgtcagatttcttttcccttctctatacaaaattaaaagatgctttcttttaaaattctgtgttgccatgacacctggttccacctgaacttaacttttctcaaatcttgagccaaccaatgcatttttcttatggaaatgtttttcttaagctatgttaatgactatgtatttaacccctagactccgtgtttcttcaagtcggtttcacctaagactcagaaccgataatgactcaacaaaccagtatgttttactcatacagttgttctcttaatctatgttaatgagactgtgtatttgattggaaacctgcctttcttcatgccaatcgtcttatggcccaggaagattcaccttgtgccaatgttatctcaaaatgcatgttgtggctgagtggcctgcagccactctctgaattttgatacatttcctttctctaattagtagcctgctgatatgtatataacttactgctgaagactagcaggggggcactcttcctgcccccttctcttttcctctggaggccaagaactctggtgtctttcattgctcagcaacaacctttcaataggaggatactgatatttctccaagaaaagctatagttttgactatacagacctttgttggcaaagtgatgtctctgctttttaatatgctgtctaggtttgtcatagctttcttcccaaggaacatctttttttttaatttaatgactgcatttttaatgttgttatgctttggtccagctgttgcattctttctgaagctattagtaattaccctctgctctttatcagtagcttattcgacactttctgacctgaggggctcatcttccagtgtcatctatttttgccttttcataacatttatggggtttgggcagcaagaatactggaggaaattcccatttcctccttcagtggaccatgttttcccagaatacttcaaatgacctgtccatttttggtggccctgcatggcatggctaatagcttaattgagttatgcgagacccattgccgcgacagtgctgtgatccatgaagagacaggaagctcttagaattactttctttttaatgcattctatttattcccctcagctagattctaagtgtaatttgtctgtttattcattgatacatttaacagatgtagaagagttccttttgtttctaaaatattcaaaatatttctttatatataaaggatattcatgattttgtaataattttcacaaactgacataataattttactgttcagttcagttcagttcagtcgctcagtcgtgtccgactctttgcgaccccatgaatcgcagcacgccaggcctccctgtccatcaccaactgccggagttcacccagactcacatccattgagtcagtgatgtcatccagccatctcatcctctatcatccccttctcctcctgccgccaatccttcccagcatcagagtcttttacaatcagtcaactcttctcatgaggtggccaaagtattggagtttcagctttagcatcattccttccaaagaaatcccagggctgatctccttcagaatggactggttggatctccttgtagtccaagggactctcaagagtcttctccaacaccacagttcaaaagcatcaattcttcggagttcagctttcttcacagtccaactctcacatccatacatgaccactggaaaaaccatagccttgactagacggatctttgttggcaaagtaatgtctctacttttcaatatggtatctaggttggtcataactttttactgtagactacttttttttttttgagatggcaagaatacacagaagaactgtacaaaaaagatcttcacgacccagataatcatgatggtgtgatcactcacctagagccagacatcctggaatgtgaagtcaagtgggccttagaaagcatcactacgaacaaagctagtggaggtgatggaattccagttgagctattccaaatcctgaaagatgatgctgtgaaagtgctgcactcaatatgccagcaaatttggaaaactcagcagtcccacaggactggaaaatgtcagttttcattccaatctcaaagaaaggcaatgccaaagaatgctcaaactaccgcacaattgcactcatctcacacgctagtaagtaatgctcaaaattctccaagccaggcttcagcaatatgtgaactgtgaacttcctgatgttcaaggtggttttagaaaaggcagaggaaccagagatcaaattgccaacatctgctggatcatggaaaaagcaaaagagttccagaaaagcatctatttctactttattgactatgccaaggcctttgactgtgtggatcacaataaactgtggaaaattctgaaagagatgggaataccagaccacctgatctgcctcttgagaaatttatatgcaggtcaggaagcaacagttagaactggacatggaacaacagactggttccaaataggaaaaggagtatgtcaaggctgtatattgtcaccctgcttatttaacttatatgcagagtacatcatgagaaacgctggactggaagaaacacaagctggaatcaagattgccaggagaaatatcaataacctcagatattcagatgacaccacccttatggcagaaagtgaagaggaactaaaaagcctcttgaggaaagtgaaagtggagagtaaacaagttggcttaaagctcaacattcagaaaacgaagatcatggcatctggtcccaccacttcatgggaaatagatggggaaacagtggaaacagtgtcagactttatttttctgggctccaaaatcactacagatggtgactgcagccatgaagttaaaagacgcttgctccttggatggaaagttatgaccaacctagatagcatattcaaaacagagacgttactttgccaacaaaagttcgtctagtcaaggctatggttttcctgtggtcatgtatggatgtgagagttggactgtgaagaaggctgagcgctgaagaattgatgcttttgaactgtggtgttggagaagactcttgagagtcccttggactacaaggagatccaaccagtccatcctgaaggacatcagccctgggatttctttggaaggaatgatgctaaagctgaaactccagtactttggccacctcatgtgaagagttgactcattggaaaagactctgatgctgggagggattgggggcaggaggagaaggggacgacagaggatgagatggctggatggcatcactgacttgatggatgtgagtctgagtgaactccgggagttggtgatggacagggaggcctggcgtgctgtgattcatggggtcgcaaagagtcggacgtgactgagagactgatctttttttttgtagactacttttaattcaaagaaatgtccgtcaattattttcttatgatcacctcaactttgtatctatggtaagcacagaaaagttcaaaactttacctcagcatttcctattatatttcttccttgtgtataagtcaataatatgtgattctagccaaatgcacaaactgttcagtcatcaaagcacttactaggtgcctgatactgcagtaggcatgatgggaagcaacatacatgcatcacagagggacatgctaaatattgttgatatacattaaaggaatagttagggaaaatatcgatataaaggaaatggtaaatctgatggagtttatagaggattgtgtattgtcaaatacagatgtcaatttctataagtttaacatataaaaatggagaacaaagggctacaatgagaagatataaacattaaaatcatctagcaaatgttatctcacaattaaaaaataccttctgtgcactaaagcactaaacttatttttcttaaagcccttgaaattaaaggctataaaccagttctttcaaagggtaaacaaaattgataaactttaagccagactcatcaagaaaaaaaaagagatggaaaagaacccaatgaataaaatcagaattgaaaaaggagaaggtacaacagatataacagaaaccagtggactataccccaatcaaatgaaaaccctagaagaaatggacaaattcttagaaatatacaatctccaaagactaaaccagtcagaaatagaaaatatgaacagaccaattaccagtaatgaaattaatcagtaattttaacactccccaaaaaataaaagtccaggacaagacggcttcacaggtgaattctatcaatttaacaaacagttagcacctatatttctgaaaccatttcaaaaaattacagtgagagaaacacttccaaacacaatctaaatgccaccatcaccttgatattaaaatcagaaatatgccacaaaaacaaataaaattacagtccagtaacactgatgacagagaaggcaatggcaccccactccagtactcttgcctggaaaatcccgtggatggaggagcctggtaggctgtagtccatggggtcgctaagagtccgacacgactgagcgacttcaccttcatttttcactttcctgcattggagaaggaaacggcaacccactccagtgttcttgcctggagaatcccagggacgggggaccctggtgggctgctgtctatggggtcgcacaagtcggacacgactgaagtgacttagcagcagcaacactgatgaacatatacacagaaaccaccacaaaatacttgcaaaccaaatccaacaatacattaaaaacaccagacaccatggtgaagtgggatttatattagggatgcaaggattttttaatatctacaaatcaatcattagaaaatttgaaaaatgaaagcaacctgattatctcaataggtgagataaaaaaaaaaggttttaaaaaattcaatccccacttatgattaaaaaaaaaaccaataaaaggacatgtggggaacctacctaaatatgataaggatcatatacaacaaactcacagcaaaaatcattctcaatgctgaaaattaaaaggcatttcctctgagatcaggaaaaagacaatgatgttcattctcaccatttttatttagcatagtttgggaaattctagtcatgggaatcagagaaaaaaattaacaaaaagaatgcaaattaaaaaagagtaagtatggactctgttgcagagggagagggtgggaagatttgggagaatggcattgaaacatgtataatatcatgtatgaaacgagttgccagtccagattcgatgcatgatactggatgcttggggctagtgcactgggacgacccagagggatggtatggggagggaggagggaggagggttcaggatggggaacacatgtatacctgtggcagattcattttgatatttggcaaaactaatacaatttgtaaagtttaaagataaaaaaattaaaaaaagagtaagtaaaacaatcactgtttgtagatgacatgatactatacatgtgtgtgtactaggtcacttcagttatgtttgactctttttgatcctatggactgtatcccaacagtctcccctgttcatgggattctccaagcaagaacactggagtgggctgctgaaaccttctgcaagggatggtcatgatctagggactgaactcgcctctcttacatctcctgcattggcaggcaggttctttaccactagcgccacctgggaagacaaatactatacatacaaaatactaaagacaattccagaaaactaaaacagctaatcaatgaattcagtgaggtttcaggatatggaattaatacacagatttcccttgtattctgatacatgaaaagtaaaagatccataaaaaattaaggtaacaattccacttaccatcatatcaaaaagaataaaacacctaggaataaacttacctaatgaggcaaaagacctatactcagaaaacgataagatactgataaaagaaatcaaagatgatacagatggagaaatataacaagtttttgagttggaagaatcaatgctgttaaaatgactgtactacccaaagcaatctacagattcaatgccatccctgtcaaatcaccagtgacatttatcccacaattagaacaaaatattttttacactttgtattgaaacacaaaagaacccaaagagccacaccaatcttgtgagagaaaaaaaggagctgaaggaatcaagcttcctgacgtcaaattatgctacaaaagaagagtcatcaaaactatatgatactggcacaaaaacagacatatagatcaatgacacaggatgagaccccataaataaacccacattcttacggccaattaacctatgacaaagaaggcaagaatatacaatgaagaaaagacactatttgcaataagttgtgctggaacaaattgacaactatatgcaaaagaaaaaattagaatattctctaacatcatgtataaaaataaagtcaaaatgggtcaaatacctaagtataaggctagatacttgaaaaatcttagagtaaaacacaggtagaacataaattgcagcaatatctatatttagatatgtatcctggagaaatgaaaataagaaaaacaggcaaacgggaccaaatgaaacttaaaatcatttgcaaagcaaaggaagccataaacaaagtgaaaagacaaaccagagaatgttagaaaatatttgtaaatcaaatgattgataagagattaatttccaacatatacaaagggcacatgtagctcaacagaaaacaaaacaacacaatcaaaaacagactattcagttcagttcagttcagtcgctcagtcatgtctgactctgcaaccccatgaaccacagcacaccagacttccctgtccatcaccaactcccggagtttacccaaactcatgtccattgagtcagtgatgccatccaaccatctcatcctctgttgtccccttctcctcctgccctcaatctttcccagcatcagggtcttttcaaatgagtcagcccttccgcataaagtagccaaagtattggagtttcagcttcaacatcagtccttccaatgaacacccagaactgatttccttcaggatggactggttggatctgcttgtagtccaagggactctcaagagtcttctccaacaccacagtgcaaaagcatcaattctttggtgctcagctttctttatagtccaactctcacatccatacatgactactggaaaaaccatagccttgactagatggacctttgttgacatagtaatacctctgctttttaatatgctgcctaggttggtcataactttccttccaagaagtaagagtcttttaatttcatggctgcagtcacatctgcagtgattttggagcccaaagaaataaagtctctcactgttttcattgtttccccatctatttgccatgaagtgataggaccggatgccatgatcttagttttctgaatgttgaactttaagccaaccttttcactttcctctttcactttcatcaagaggctctttagttcttcttcattttctgccataagggtggtatcatctgcatatatgaggttactgatttttctcccagcaatcttgattccagcttgtgcttcttccagcccagtgaagaatagcaaggagcgataaagccttcctcagtgatcaatgcaaagaaacacaggaaaacaatggaatgggaaagactagagatctcttcaagaaaattagagataccaagggaacatttcagacaaagatgggctcaataaaggacagaaatggtatgggcctaacagaagcagaagatattaagaaaaggtggtaagaatacatagaagaactgtacaaaaaagatcttcatgacccagataatcacggtggtgtgatcacccacctagagccagacatcctggaatgtgaagtcaagtgggccttaggaagcatcactaccaacaaagctagtggaggtgaaggaattccagttgagctatttcaaattctaaaagatgatgctgtgaaagtgctgcactcaatatgccagcaaattgggaaaactcagcagtggccacaggactggaaaaggtcagtttgcattccaatcccaaagaaaggaaatgccaaagaatgctcaaactaccacacgattgcactcatctcacacgctagtaaagtaatgctcaaaattctccaagccaggcttcagcaatatgtgaactgtgaacttcctgatgttcaagctggttttagaaaaggcagaggaaccagagatcaaattaccaacatccgctggatcatggaaaaagcaagagagttccagaaaaacatccatttctggtttattgactatgccaaagcctttgactgtgtggatcacaataaactgtggaaaattctgaaagacatgggaataccagaccacctgatctgcctcttgagaaacctgtatgcaggtcaggaagcaacagttagaactggacatggaacaacagactggttccaaataggaaaaggagtacgtcaaggctgtatattgtcaccctgcttatttaacttatatgcagagtacatcatgagaaatgctgggctggaggaagcacaagctggaatcaagattgccgggagaaatatcacctcagatatgcagatgacaccactcttatggcagaaagtgaagaggaactaaagagcctcttgatgaaagtgaaagaggatatggcatcaccgactcaacagacatgaggttgtgcaagctccgagagttggttatggacagggaagcctggcttgctgctgtccatggggttgcaaagagttgaccatgactgagcgactgaactgaactgattaattagtgatattcagtatctttacatatctttagtggccatctgtgtatcttcttttgaggaatgtttatttagatcatcagtccatttttggcattgcctttctttgggattggaatgaaactgaccttttccagtcctgtggccactgctgagttttacaaatttgctggcatattgagtgcagcactttcacagcatcatctttcaggatttggaatagctcaactggaattccatcacctccactagctttgttcgtagtgatgctttctaaggcccataaccaaagctaaaacaacactcaggtgttgatgtgactggtgatggaagtaaagtccgatgctgtaaagaacaatgttgcctaggaacctggaattttaggtccatgaatcaaagtaaattggaaatggtcaaaaagaagatggcaagaatgaacatcaattattttaggggtcagtgaactaaaatggactgtaatgggtgaatttaactcagttgaccattgtatctacttactgtgggcaagaatcccttagaagaaatggagtagccctaatagtcaacaaaagagtccaaaatgcagtttttgggtacaatctcaaaaacaacagaatgacctctctttgtttccaaggtaaacattatcacagtaatccaagtctatgccccaaccagtaatgctgaagaagctgatgttgaacggttctatgaagaccattatggagaactgccccaggagccctgactctccacgctttgtgggtgctcctatcggagacagggcaagttgagacatagctagagaaagacctgaggcagagacaagagatgcaggccttgaggtgggaaggtgtcagtgttctggaagcctgcaacaggtgaactcaagtgggccaagaaaatgcaagacgaggtctcaacagcatctgttccaagtctattgagaggtacacaaaacaatctgagtaagctgattcatgttattttcctggatacgcaaacaatgtcttagctcaggctgctatgacaaagtaccacagactgagtggcttaaagaacacaaacatttctcagtatttatcatgctttgaattctatttgtgcaatgtttgaggggaatgcagtgcttatctttttaataggtacattaactgtgtcttcgtttggatgtaccaaaggatgagacaatgggagatgtgagttggtggatcgatgcctcagcttccccttcttgcagctggatgatctgaggtgtattcccattatttcacagatggtccctgtggcatcaagctccactcacctaccatggtaatcggcccacttttcctgactttgctcccttccttgtctcatgttttctacttcctcactttgcttttctggaggctgggaaaaataagatcttggtattggtcaatccagttactggtgaggactctcttcctggtttgcagacagctgccttcttgctatgttcttatttggctgagacagcaatcatctctcatgtctcttctcataatgacactgatacaggagatagatgggctccagtttagacatttataactggcctcctgtttgcattttatggggcagaaaaaagtgggcttcaggctggacacttacaactagccttctctttggatttcctggacaaggataggtgggctctgggtaaggcacttacaaccagccttctgtttgctctccaaaatggaagtaacaatagaaacagagtaaatagccagattttgtctcttgtcaatatcttaaaacaatagtcgtggcaagaacaaagaggggtaaaatcctatttgagtaaaggattaaaggctctctgctcccctccttcttgggacaagggagacactacacatgcacagaaaggctacttgggagacaaaagtcagaggaaatgccaggccataatgagtttcccctcccaaatgctttcaagtcagttcattttggctgaggggtgcatgaacacgtaaggggagggtcctgagacaaattagctggggggacaaaacaagatgattagcctgagggaagaaaaagacctggaaaactccccccttataaaggatttaaacttcccaaaggcatgactcttctctgagcttccctgtgcatcttttcacatgtatttttccaataaaatttttacttttctcattaccttctgcctcctcacctgaattctttcttgacgagacaggcatggactatcgacccaggctctagccaactggcctttgtggtctaatggttaggactcctgatctgggaactaagatcttgctccctgctactgctctctgctgcttgctgcaaggggttgcttactgatgctagcatctgaaatcaacactaagctcatccatgagggttctaccctcacgatctaatcattcccaaaggtcccatcttcatactggggataagggttcaatgagtaaatttgggagggacacaaacattcagtccacagtacataaagatgtcaatcttgcacatatttatttctaaatacaatgccattccaataaaaattccaactacaggtttcttggaattcaacagaattatataattaacctggacagaaaaactaagaatattagagagattctaaagcaaagagaggatagtggttgatattagtcccattaagtactgaggcatatgataaagcttttacccacttcatgtattagtatttatgtgggtttcctagagcatgaggaaacaaacctcttcccatgtagcaccccttcctccagttgtaacaacttgaattactctgattaattgtggagctatgtaaaaagaaagcaggtatatccaccaagtagggtagcagcttactgttcaccaatgtgacactgtgctttacaacagtcatccagaagatcacaatacatttctcatatatatttagtcatctactgctcttgattcacagctcccaaaatctgcgggatttcctgagcaataagagcaagaacaatgtgagtatcttttgttataatactgggtctcttccctcagttcctaaaatcacttcagagccataaaggtgtcttgttattaatgcaagtccctttccacaactactgaatttatgttagtgtaatcacttttcagcttccctggtgactcagttggcaaacaatctgcttgcaatgcaggagaccacctgtaatgcaggagtcctgggtttgttccccaggtcatatcccctggagaaggaaatggaaacccactccagtattcttgcctgggaaatcccatagacagagaagcctggcaggctacagtccataggggtcacaaagaggtggacactatttagcgactgaaccacaatcaccacaatgacttctggaaagcacctaaggatgggtggctaattgccagttgccaggggggacaacctggcagaattgacagatggaacttaagttctagcccatgacttttgggctggggtgagatactagaagttgaatcaattaccaaccaccaataatttaatcaatcaactttatgtaatgaatcctccataaaacccccaaaggatggctttggagcatcccagttgtgagcatagagactcagtcacatggacgactccacacggtccctcaatcccttgctccatgcatctcttcatctgactgtttctgaattacaactttttataataaaacaggattgagctatagaactcacaaaaatgtgtttttatgagttctataggcagcatattaaaaagcagagacatcactttgtcaacaaaggtccgtctagtcaaggctatggtttttccagtggtcatgtatggatgtgagagttggactataaagaaagctgagcaccaaaaaattgatgcttttgaactgtggtgttggagaagactcttgagagtcccttggactgcaaggagatccaaccagtccatcctaaaggagatcagtcctgggtgttcgttggaaggactgatgttaaagctgaaactccaatactttggccacctgatgtggagagctgactcattggaaaagaccctgatgctgggaaagactgtgggcaagaggaaaaggggacgacagaggatgagatagttggatggtatcaccgactcaatggacataggtttgggtggactccaggagttggtgatggacagggaggcctggcatgctgcggttcatggggttgcaaagagtcagacatgactgagcgactgaactgaactgaactgatgagccactctagcaagttaatcacaggaaaggaaggagtcattggaacctccagtctatagcagatcagtcagaagcacagatgacagcctgaacttacaactggcatctgagtcaggaagaggggctatcttatgagactaaatccttaacctgtaggatctgatactatctctgggtagatagtgtcagaattgagttgaattgtaggacttgcaataatgttggaaaattgctcgtggcagggaaaccaccaccactcctagacacacacacacacacacacacacattgggtttgagtgttagaatcattttaaccagtgataagaaagattaccaatggtgcaggaactgccagtggaaacacaaagtctcctgaccaaaacagagcaaatcagaagccaaacctgggttgagaaacaaagaaatgatgacaaaaatcagacagtttgtttcgaaaatctgggaccaggaagaaattaagtgaagagcccaagtgtggcaaaggttcagggtgacactaaactttgttttgcatacctgggaacttcacacatggctagacagaaagagaaaaggacaaattctgtgggaaccaagggggaaataggcacagcaagacgaggacatgggataggtaaccacactcgggacattgtttaactgattcttctggtagactctcatttgcaaggtcctgaggctctcctcccaagcaatttataacctagttaaatacggacagaacagcttaaaatggtgctggatgataccagccagttgctgctggagcaggaggaagacagttgagttttgttggggaaatatggacagcttcacagaagagaggggtgtgaaggaattcggggtgaggaaactgcttgagcagagcttcagccaaggcaattaaagaaggagcaactggcctcgcaggactgaagcccagcggggattcagggagaaaggttgtcagaaacatggtctgggctttttcacacaatctgaaccccagactctggagtctagccactaaggaatcattttatgtgtttaaagaatggagagatatagccgcctgtaatacatctgcaccatgatggtttctctttaaattcacaatcattcatccttagaaaaaatgtaatttctgatgcaatagcttaaattgctgaactgagaaagtcagaggggagaaagggggaggaatgaggtctttgaggacatgaataccctcaatgaatgggcccatcctaaagaggaccctcattattagagaaagtttaaaatactctccattacacccatactctcctccagctaatacctatttctctgttcctcccaggacaaaacttctcagaagaattgtctctactccctcaccttccatttcttcatcaatttactctgtctgttcttatcactctcttgaaactaatcccatcaaggccccagtaacgacctccacgtcaccaaatccagtgagttctttcccacctccattctattttgtctctctatagctgactcctgccttcttgaaaggcacctcctctctcacactcttgatatcctcctacttcactggctgtcacttttcagtctgtcttgctatcttctctttctctaattcaaagtgtgactcagcagatcagcaacatctaggaacccaggagaaatgcagattcttggacttcatctcagaattactaaatcagaatctctaaggtgggcccaaccagtccatcctaaaggagatcagtcctgggtgttcattggaaggactgatgttgaagctgaaactccaatactttggccacctgacgtgaagagctgactcattggaaaagaccctgatgctgggaaagattgagggcaggaggagaaggggacgactgaggataagatggttggatggcatcaccgactcaatgggtaaactccaggagttggtgatggacagggaggcctggggtgctgcagtccatggggtcgcagagtcggacacgactgagcgactgaactgaattgaactgagggtgggcccaggaatctgtgttttatccagaccccaggtgatgcacactgaaagctaagcaccatgatctaaaactgtctgtttcttaccattcagtcctgattcctcttctttcctctggctacactttctttcttgatgttatcatctaagcccacaactttgaaaatcatctaaatactggtggtggtttagtcacctcagtccaactctttgcaaccccagacccctctgtccatgggatttcccaggcaagaatactggttgccatttccttttccaggggatcttcccgaccaagggatcaaacccgggtctactgtaatgcaggcagattcttactgacaatttctaaatgcacatcttcagcaccaaccctcccaatctggagattacattaaaattcaacataccccaaatggaagaaacttctctaaaaatggttgcgtctaaaacttggtcttcctccatttctcaccatttccgaatacatcctctaccaccaagccccatcagtgcccctcaggccccacacaacaatcagaatggtctttgaaatcaggtcaggtcaccaccctgcttaaatcttcaagggcttcctatcacatttaattaaaatctaaagacctggtcattggctgttaggccctacatgacctggtcactttctatgtggcttcctgtattccctttgttgtctaatgtcagaaactataactatctagttcacactaggttctctataaattatttgctgaacaaaatatttcttcttttgaaaataagagaaacatagagtttacttcgttagcttctccacatttgctgaggaggatctatgtgatgttgacaggtaacttcaattgagccaggacacaggagatgcgaagggagactttcaaagaatgtcttcatggtgccataacctcagcacagccaggttccagaggacaaacccccaaacatgcttgtcattcagttcagaatgtagccttccttatatataatgggatatagtagttgtggtgatggtagagactaagatgaggaatgatgtagggccatttgcaaaaggtttctcctgtgggctgaccaacgtgatgttgttcatgaggccagtgaaagcccctaagaatctacaaccccataatggcagttttcaaaaatggccagaaattctttgatactcttcccattgagagatggggtccatgattcctgcccttgaatctgcatgggcatatggctactttggtcaatagcatatagtgaaagtgatgttatgtgatattatgtgacttttgagactatgtgagaagcggcaatgcagcttccatgttgtttactgacagtctcacacttgggtcctcttgggacttcttaagccaggtaagaagctcatcaaacttgagactactatgctgggaggaagccaggccacgtggggaagtcacgtgaaggcacttcaatcagtacacctgattttcaagtcctcccagcccaggtgccagccatgtaagtgactgaactaattccaactcctagctatcacgtcaagcctcagacgtcatggggcagagtcaagtccccattgtgcctgtccaactctttggcctacacaattcatgggcataataaaatggtggtttctttaaaccattaagttttggagtagttgctacatggcaacaatagccagaataggacaaaaggtaatgtcatttcgttccctcaaaccctcacgaactatattgccttttgagcattttcttggttgggggagggaaggaaatcattcagccagttgacattggattcttttgaggaaaaaaggctgagttttggcatcctctaaaggagctgtacattgcccctcctagcaggggaaagtcagtcccttgcccagcctgatctgatcactcactcccagctccacccagctcaagactcaaagagatgcttcactgcccccaatgtgcctcacaataaacaatctctgaggaaagaaggtaaggctctaaaagtagtgtcaacttaatcattatgtaagactgactggatagaaaacagccctggcattgctgcctaatactcatttctagctggccacaatccatttctgctactgaatcatcatcatcccatcgtcatgttttatagacctctcatttctccttcccagcagaactttcaggcccccatccacattcatatattcatcacctcattctacacaattctcctggcctctcccctccctcacttgtccccatcctatttgagagatccaccctgagatatttaccctaatgggtgtctcaatatttttaaacctctttccttgcaagcttagtggagcctcttgcccataacaaggggactagatatttcatttttcccaggtttatacccattgccctggcataattaatattggtactctcaaaagtgcacaaatttgggtaatgatatatatgatccctctaaccctaaaacatgtcttctatcacttgccatccttcacatgagacaaacacctacataaaattttggcagtaataatgatcaagtacacaccatgttttatacaagaaacctcaggtaatgtgcagaatggacttgttaaatggagtgcatttccttcacttatgaatatcataatctaaatcatttattttgtagataatgagcaggaactgagtaaatgacggcaggtgatggctaatatactttctaggcctcaaattttaatctgaaaattcacaaacattgggctcaatccagggcaatagaatttttgtcccttttagaaatttctggttaccaaagttccagaaattgctttctcattccctaatctttcattttctccattacgtaacgagaagctggggctttggccgattttccctttaaagatgatttttatcgtcaacaagcaatttcagggagtgatgagccggggaagcggtattagctgatgctagcgtttaagctagtctcaactcgtttttcccagggacttagattcctgggtctgccagtaaaccccgggcgccggcagctggtgcgcctgagcgtgcgcgcgcgcgccgtcgcctccccgcccctgcccctcctcctccgcccggcgactcacccgccctagttgccagtcgctgacagccgcagagctgagagcgtcttctctctcgcagaagcaggtaaatagccgcgtagtcctttaaactcccagcggaggacgcccaaccctgggtcttgcggccgaggccccagggcacccagccgaatcggattggtgggaggcagaccttgaccgtgagtagggctgggggcttgcggcgggcgcggggaacgtcgggcctgttgagcgtgctcgttggtttttgccagccgccgctcggttttaccctcctggttaggagagctccatttactcggaatgtgggctggctggtccccctcccgaggtatgtgggtggtgtgtaggaatctagccccctcccacgctcgtccactgcgggagtgggatgggcgaatcgcaccggtagaggagccgcaggtccgaggaaccgctggggagctcagaagaacaagggcgaggccccgggatttgggccctcccgaagcccagaggagtcgcggaattgggggtgggggtggtggggaagaaacgggcgcccaacggggcccgacctcggcggtgaggagtgccggagcgtccgtgggcccccagccgctgctgccgaactcctcccgagaggcggccctgcctgccatcacgcggctgggaggtacctgggtagccgcagcgggtgggtctctggcaaccccccggggatcggctctggcgggcgtgcgtggcctgggcttcagcctcggcgcggggaatcatgggccacctggcgctctctccgggccagagaaatccaggtaccgggaacagtgtttcctgggagctctgatgtggtggacccaaaagcaaagcgaaattttccctgtctcgactgatcctccggaaggagggagctcggccgtcgggagactgaggggaggggatcaggcgcctctcggagaaccaccctcatctgccagtgagggtggcaccttcacgcttgatttttttttttcccccttcacacgtttgattattaaacaacgagaagtccgttttttgctgtcctttttccgtttttttttttttttttttccttttggtaccatatgtagcaaatagatttttttaaaatcataagcccaccaccctcaccatctttttttcagtttcctcgtctccagattcttaacaacaaagcagtttcacctccctgatcatggttatccttatctcatggccgggttattttcttgtacttaagagcaatcacgttttattaagcagttccccgaatgctgaacctttgaagtgttacctttccttacaaaagataccacatagaataggattaaaaattttcacaagttgtcagagaaaaataggaacagaaaattgtataaaaatgtcagacctctggaaaatgaacagctctctcagatttgaaaattaacctatgaaaaggaacagttttcctacggaaacattgaggtgctctaacaatgaaaaagaatcagaaaaggaaaaaaacagagttaggatgtgatttgtatatgatttgtatctgatgcaaatttttcatacttgtgaaagaaaaatatcaagattataaaaagataaatggtgaaatgaacaatcatttatgaaataaaatacaaatcaaagcaagtctggatttacaactactagtaaaaacaacagtaacagcaaccacttctggaaagttacctagaaatttgcatattcagtatgtgaggtggcaaggctttggagttagaaatatggctctgcaactaattttacagtttgggacctaatttcctcatcccccttttggacattcataaaatagaggaaattatacctacttcaggagtttgccaagattaactgtgtaaaactgacctttagtgtgtatacttttattcttttcctagtcacactgcactgggggacgttgtgaatctgtatgaaatttgtgaaaaacagtcaggtgatcctttaagccatgaccctaaaaccccactcctgggaacttacctgtaatggaggaaaccaggaaagaagaagaaaagctgcattcacccacagaactcagaatgatctaaaattagatccagtccggagacaacctaaatgtattaataaaatagcagggcagcagctaagaaaatcatagcactttaactgaaaggaacattgtgtaaccatcacgagtcataattttagagcctctctgtgatatacaggaaaaaactgacaggtcaaagtaagattactcagacatggatgcgtttgtggaaaatctgaatgaaaaatgaatccacagtttgctgtgtatgggaggagagttcagtgtcacgtttgctgctttttttaagttagcatcatctcttttttaaaaatactatcatattttttccctgagtagattcattagtggtttaataatttatatactgttattctgttaaataatccgttcttagatttatcaattatagttttttcttttttttttaaggacttctgaatatatttgaaaactgaacagtttcaaccaagccgaagcatctgtcttcccagagacacaaatccaacttgagctgaatcacagcagatgtaggtaccctgcagaatctctttggtcttgtgatggttgaaagtgcccaactgtttcacagaagataagggactgaaaggctgggatcacaaatccttgctgtggaggccactgaaatctatatatgtaacccacacctattatatcactctttcttgtaaaagcgtcttgattttgcagggaaagggacatagctttctctggaatcattctgagttatgtaagaagcagccatttaaaaaatagtataataaaagcaattacctaacatttctgcaccaaatcaacactgaaggtgactatcaacagacaaaaggtttatgaggtaatggtttttctaagctttagttttaatttacctattccattctccctttttagatcttatttccttttccaaggcagccagtttatcaactgtgaactgctgcatatgaagcattcaaaacctgactgtgtctaaagctgtgatggctacagcacaatcatctttgagtgaatagtatgtttaacagttcttacagttgggagaattttttctcagtttgttcatcctttttctcctaaccgtgttctgcttattgctgttctaatattgtgtgatcatgtcaagggaggtgttcccttttatgcaaaacattatgttaaatgttgtcttcccgagaccaagctcggaagattggctaggagtgcagttccgtgggaagccttattataggttcctaaatctcatcactagatactcccaggctgttggcctgatgcagactctagctatgttgcttttcttaaagctcttcacatcactctgaggatggactagactggggaccgtttgcccatttcagtccagggctaggcctcagtgtcagtagaaaaacctccacctcaaaatggtttgtaaatttttgtatagtttgcattagactcttgttaagggacagtgacctcaaaagatgaaaatatgacaaatgagttccacttagcttatgaaaaattggaaatttccccagggcaaggatgggtagagggactgtttggtgccagtttccaatttaaataagtctcaagggtataacatattttgagtatcaaaagtgtggcccctggcacatgaccactggacataagttcctaccagctctgattctcaatccccatgtataaaagggaataagatgaatgggacaatatatggattttgttgttgttgttccttctctctcgttccatcgctctgcctttgtgcttatgcactaatgccacgagattgtatttattatagttttccaatccattctgatacttgccaggccaagtataccttagatgttcttcctgttcagtaatttcttcagtcttcttaattttgagtatcattatattctttaaaatcctctttgagttaggactgaaattgtattgacttaatgattaattggagttgaattggtatctttcaaaatcttcgatcttgattttcccaaccataaaccttgcctgtctttcttttctttccagtcttccagcttttttcatctaagttctactatttattattaggttaaatcttagtttgaattttttgttgccattaatgagtggaatttttttttcacactaaatcttctaataattacaactaattggagtattcactcttttctatatgttgagttcaaaaactgccaccctaataaataagctcattgatttatttgagggcatttttaaatgattgtcctggattttccagatagaaaaatcatacctggctttctccatagcagtctaaaatgcagcaatcacttacattcttgtcttgttaatctcattcacgagaatgcttttcctctttctgttaagatagggtgtggaatatgtattacttctagttctatagaaattttttcaacatcttaaacttaaaaagtcaggaaagcactgattcctgagtttaagtgagaactttgattttaattgaaaactttgcaacatcagagaatcttttttttttctccttagcctactaataggttaattgatttcatgattttgagccattcttgtattcctaaaataatccttattgtcacagtgtattctttcactaaaatatcaaaatcaacttggtggtattttcttttggattttggcacccatattgctaaaggttgggtagctaagagtgtgagcccttcactccctgtggctcttaatactacatctcaggtgaactgcccaaatgtttatctctactgagtaagagttcgagattcttataacaattgcctaaattgatactctcacttgaatggctcatagatatagcaaagttaatatatccaaactatagctttattttttctcacaagcctggccctccattagttttcttgtttgcattaggtggcatcaccaccatctacctagttccaaaagccacaaacctgctcaactctcactctcccatccactctatcagcgtaatcgtttttttctacaaaatacttcccatttttcacagctccgtgctgctttgtcacagttccaggttacaccaccatcttcctttaatcattttctaagtggtctcaccatttcctttcttatccctcaaatttttctctttaacacagcagtaagggtgaactttaaaaaaaaaaaatctgtgatgtgattctcctaagccactttaatggctttgcactgtttcaggctccagacacgtatctccctgtagtcctcaaacccacaaactaaatatcttaccactgtgtcctcagccacagaacagtaatattagaggacatgtaataaatatttgttaagtaatcaatagttattgtacattgtacattgtacatactagctgttccataaatatttatttaatttaaatatttatttaattgaatgaattcaatatcagtttttataattgaatagaaaagtaatccctcccacacagatttttctttctttttttttttttttttttatgaagcaaggaatatgactacttagaaagctggctgccagagaaaatggcagactaatgtcttaaaacaaatatcagtgtctagatgccaggttcttttatagaacagagatggcaggaagatgaggaaataaaggcagaatagagagggagaagtggggtggaagtaaagtaaaaaaggccacgtcctgcaagacatctccagaaaggccagcctgtggaagggatgtgttaatctcttcttgcctgcaaccattcacaggtgggaagtgtcaaattatctccctgtgagctgaacaaaggcacttcagtccaacagttagagagagggactgggttttctgaggcaggctattatatatgattgtaacaacaacagcaacaaaaagcaagtcaaagaaacagttccaatatggagtcagaattggttcttctcagcaacagttccccactgtcaaggtccatttgacaatcttgtaggaaaaggggacagtgatctttctgtttgtcagatgaatctttctgggagtgtctgcctttgatgccagtgttccaaatagtgagatttgttttctttttgttcctccttggaaagtgtctactttatacttgtcaacttagaaatattggacttcaaacccaggagggtttctcaagttaagaaattttgcacttttctgtgtatgggaagatgcaagcgtcagggttcattaaaattatttccttgatgtgtaattcagctgtctggggcctgtgatcctgtattctcaagagtttcctcaaggttcaccatagggagtgggtgcaatctgatgactgctggatggcatgtattctccttcctgagttttctccttcctgagttgacttggggttcaccagctcacattagagggcttcaattgttaattccatttctcaggtcttcccttggtcaggaatttgaccaatatttgggagacatttcatggtcaaactttgcttcacggtgctgtgaggttcatcccaaatcaggcaaaacttcttgatgtaccactctaggtgctaaattttggattagacccccatgaataattaaagattctctggattctaattgtcatccaggagacattttccattgttgcttcttcccatacctagaatcacactattataattattttatgttataaatgtgatctattttctcaagatgtttatccgtagaagttttgttggaggcctgattacacattggttactgcaagagacaactatcttataaattagtcaggatataagtaatgcagctagtaacactgataatgacatagtgcaacaggggtgtgcaaagcacaatctaaaaacaaagaataaagtaaagcaatgattagtataactagttgtagtccagttgcaataatctagtgaccaaaggagccataactgatttaatgatctatcttcagtttcattgtaccagccatgccatagcttaatctttgagacaagcatatgctactggcaggatcaaccagatagaagaagatttaagttttacttttgcttacaaaggatataatttaccaagttactgtaagtcagaggttaaggaagttttccttacacctgaaaaacagatttaaaccagttatttttagatagaaaccataaaaattataacaagttcagttcactcagtcctatgtaactaatcctttttgttaacagctttatgaagccatcaggtttcccattagaattcttcaatgtgttactagttcagcattatggtctaaaagtcagaaacttggatttatctgaaagtcctttttataaatcttcttaaagaggagacatttttacagaggcaacagagtgagaccataactgtccataatgaccaaagacttaagaaggcactttaaatctgattatgatgcaattgacaaagtaacctggttactgctgtgacatacaacagtttcaggtagtagaagtagaatcatgactgataatattctaccaggacatatcacatttttaggaactccatataatctctagtatatcagtatcatttatcatataatttaagatatattattcattggacaacacttgccatgtagtttaacataccaagtgaatctaattagtttaatatctccctttggtgtgtctcaggggccctttgaagcaccccaaagttagctaaagatcaaaggaactttattgtaacttgatttgggaagtcttgtcaaaagagcattaagaaaaaatgttttaaaacacaacaggatcataggtcactgtgaaacaatagttatttacttagccaaaatgacaataaaagatttcaaaagcaaatatagaacagattatttaaaaggtagaaataatctgttatcaaaggagaggaaagccaaatttgttttgtaccaggttactttcaagattcatttagtcaattaaaattttttaaacttagtcctgatcatgtacaaaacactttttcagggtccatgtttcacgaattttccatcacttaatttattttagcacaattttaactttcaagttgttgaatatctggagatatcctaaaatataattatttctgaaagttcactctaagctcttatcttcatttgcattttctttgcttaacagtttattcacatcaagtccctttccttgctgacaaattgtatcaacaaacaaccataaataccaaatataatttaatattaaatatttcccagttcacgtgaacctggagctcatttagcttaattgtatttagaattgtttggtttgtaagcacttacttttatttaaaccaattaaatagagctcttttacaaatcaactgcagcaatattatccaaagacaaagatacatacaaacacacaaacagagaggcctcagttcttatttcaagatttcagtcctgagtcaggcaatgtaaaacccatcagtttacatacgaggttgaattaaaattggatttctcatagatggaataagtcaagctcacttggctagatagctaaatatttgcagaaaaagcacttaggaattctaattatcttggccaacccaacttctaaattactttaccttctctaaaatttgcattttaaaaagacagcataatgagggttcctgagaggacatttgcatctcaaagacatttgcatctcagagttacaggcaagtttttccaaaaatgactttgtttctcctttaatacttacaggcctcttaagataaccagggaaggtctggggagtagtaaaaggatttatgggatttggattatttttggaaccacacttctggtgctgtaaagactacatttgtaaaacacagttttgtttttcttttttgagtgatactgaaggattgacatacccatttacctatgttggaaagttttatctttcctcatttagcttagctcttgggaagacacagaggcaacaatttaggctcctgtaaatcagtctggactgaggggggaacgaggtgaagataagaaagacaggctgaggaatccgcctcatagtcctaccaggaagatagtggccagggcgaatgggtcagtgttttgcttgaaccaatgtgtgcttcacagtgcacagaagctgtcttgctggagttgttgtagccacaagttccggcaaacaaactcactcagaaggacaatgcagatagtggagtgcagtttattacacctgcgggcccaaggcagagtctcctcttagccaaggaccccgaccagtttttgtgaaaaccttatatatcctaagtgtatgtgcccaaacccacctccccgaattccctgaaactagtctgaacaaaggaaaagaaagatacaatcaaagttaacctgtgattcatatgccttaagcctaggtagttaacagtggacagttatcaataggcctgtggtcataccccaataagcataatagaatttatgattctattcggttacacagataattagggtattcttttaggctactgagagtctaggtatgagccctggggctcttgcgggggggggggggggtctggttttccagttagtatgtcatttccatagatactgggcatatagctcaaagtccacagtccagcccaacatggagtcctgctttcaagatggagcctgttctgtctgtttcttcgttcagagtcagatgctctaatagcctggtccattctgttacccacttagcctgtcacaagagacttcaagcgacaggcaccttaatggcttttaagtgcctgacccatgccctcaaaatattaattggcctggtactcagcctaagggagaaggaggtaaggagagccctgacctgttgggaggcagctaccggggcacagagggctattaggcctttagaacaccccagagaataaccctagctagagttccatagctattagtctgtttgcagagggtttaaggagaaagggatgagggagtgtgaggagagtggagaccttaaaggaagtacttctccttttagctcaagcaattagtatcagatgtctatatgttaccaaagtatccggaataaaccaaaatctagccagctagagagtcacattaacatgacttcccagtttcattagacctgtgacctttgtccaaaatgctttataaatggagttttccttcacaggggtgcttcccaagctgaagctgaagctctccactgtcaaagttacccagggctcccgtagggaaattagaatcagatgcctcaagtccaagggagtccccaggcctcttcacttatatcagagtgttcctctcctttgcaaaacacttctaattgcaagagtgtgtaattgtgagccatttaggcccattgctcttctgatcttaacatatctaatatatgtcccccaaagcttttcttcaggatagatgaaatatttcccatttttataagtttcatagcaccaaaacacacacaaaaataggcaaattccaacataaatgacacaaattccagtaccaatacatagatgaaccagtttccagctcaggtaaataaatttaccctacaaaacaaatgaactaatcccaactgtgtgcttgttctgtgcccttcctctggacgcatgcttgctaagccgcttcagttgtgtctgactctttgcagctgtttggactgtgtcccaccatgctcctctgtccatggcattctccaggcaagaatactgccatgcccttggatagactccagtattcttttactccagtatactcattattcttgcctggagaatcccatggtcagaggagactgacaggctacagttcatgaggtcacgaagagtaggacacgactgaaatgacatagcacaaaacaagcacacaagaactagttccccaataaactggttccaactcaggtagaatccaacaacaattcccatctccaacagggtaccccaaccaaattgactagtcctgtaaaggaaaagcccaaatttagaggggaatatgttctcagtgtgcacaccagagcaacttaccctacaaaatcaagtttgtcaactcgtaatagaaaagggcacacaaaaccacaaacaaatgagccagctatcgaatgaagaaaactaatgctatgaaattgggtctgtcaacttgtagaagtttgttgattctttgcttcaactgccaggccctagggccactgataccacttcagggaatcttgaaggagagatcctcagcacaaatggtcccagcagctgctggagccttgccctaatattccctaacagagctggctaaacacaaacagcaagtcaaatttgttaccgaatccaggcttgctctactgagtgaacaacaggccagtgaatcagagatgagatgttgagggaaagaatgtgattttattcagtaagctggctgaccgagaagatggcagactaacatctcaaaataaccatcttcttgggttctggagtgccaggttctttatagaacagaaatgaggggaagtgaggaaataaaggaaaaaggcagaatagagagggagaggcaatgagtcttgggccatcagtcttgcaaaacatctctaggaatccccacacagttttttaattcataaaacttttaactttcacagttaggtctctaatccatttagagcgtgcttttgcatgtagcattaagctccaatttttattttccttcaatttcccagaagtctctgctaaataaactttcctttctcattgatttgttttgtcaatttatcatttatccagtttggactaaagtcaatgtatgtggctttatctctgaacttgttattctgcttcctttgatctatatgttcatttctaggttgttaccatatttattactatgactttatactaggatttaatgtttgataacagtaggttctcacctcatttcctttctaaggttgagtttgttatttgtggacatttcttcatcctttattgaattcctcaaaaaatccagctacaattttgattgtcattattattcatattataagttcatttgtaggaaattgacatctgtataatactagggggttccactgagacttttccatttttacagatcatcttctttgttctttagtagtgttcaatttctttttcccctagtcttactttctcttgaattaagtcaatcctagatactttacagtatgaaagtgaaagtgaaagttgtatctgactctttgtgaccccatggactatacagtccatggaattctctaggccagaatactggagtgggtagcctttccttgctccagggcatcttcccaacccagggatcaaacccaggtctcccacattgcaggcagattctttacgaggtgagccacaagggaagcccaagaatactggagtgggtagcttatcccttctccagtggatcttcctgacccaggaatcaaactggggtctcctgcattgtaggtggtttctttaccaactgagctatcagggaagcccactttacagtatgagttgaaattattactatcttatttattaatttttttctagtcaattattgctgatatagagaaatgctgttgattttttaaaaccaatccatagccttgctgaactctagttgagtttcctgttacctctgccaagtgtgtggaattttctatgtatatgatcacattaattccaaataatgacagctggagctcttttcttacagttattgcaccagtctttgtccttgcatatggcattggaagagggcttcccaagtggctcaatgttaaagaatccacctatcaatgaggagatccaggtttgattcctgggtcaggaagatccctttgagaaggaaatggccacccactccgttgttcttgcctggggaatctaatggacagaggagcctggagaactacagtccatggggtcacaaaagagtcggacacaatctagcaactaaaataacaataatggcactggaaggatctccagtcctgtgacaggggacgtccttgttttgtttctgatcataaagggactgcattcaaaaattatctattaattatgtttaccatttctgttatataatctttattaagttaagcaggtttcctcctattcctagtctgctaagagtatttttcttagtgataggtattcagttcagttcagtctgttgtgtcccactctttgtgtcctccatggactgcagcatgccaggcttccctgtctatcaccaactcccagagcttactcaaactcatgtccattgagtcagtgatgccatccagtcatctcatcctctgtcatccccttctcctgccctcaatctttcccagcatcagggtcttttccagtgagttcttcacatcaggtggccaaagtgttggagtttcagcttcagcatcagtccttccaatgaatattcaggactgatctcctttaggatggactggttggatctccctgcagtccaagggactctcaagagtcttctccaacataacagttcaaaatctaggttggtcataactttccttccaaggagtaagcgtcttttaatttcatggctgcaatcaccatctgcagtgatattggagccccaaaaataaagataggtattgactattatcaaatacttagtatcttgatgtgctaaaggattaggcagcaacctgactattctctgagaacatcttatatgaagtgttataacacagccaggcattcagaaacctaatgtgcatttctagtacttttactgtaatcacagatatgtttcttgaatttgctaatctttgtagcataatttgtacagtgagagattttggattaaataatacaagatcttctttattttatcacaaacagaaagaaaattctccaaggtctcattaagttttgagttcttcttatttttagaaatgattctctacttcaaaaaaattttttatgattttcataattaagtgttgcttttgtgttctcaactgattttaaaatgattttgtcttaatatagtttacaattatccaactttattctaatattcaattttaaagtaatgatcagcaacatatgcccgtggccttatgtcctattgcctgtatctccagtccatggtcattcctcacctccagactcaccaagcttaccagactgactggtatctcttgtcccacgtgtagcttgataatggcattgtcaaagagacactcctcatttgtcttcctatggtcttgctccaccttccgtcttctgtccacagaattgtccaatccagaaatctgggtgccacctcaattcttctagtctttcatcttccatgtcaaacaccaagccttccagaaagttctcatgttcaattccctgaggtgtttgagcagaattgtgagcagcatggaagccaagatctgagccccatgagcaaatggagagatcagggaagcaggcctgcaaaagaccaggtgcaagaatgaggccatttggcatatcccaggaccctgttcctctggcctttacccaaaacagactccaaaaattctagtagacatagtctgagcagtttaactggccttataactttcaaatatatttttatttatacccaggcatttcaataatgatagtagaaacataaatgggatgttaattcattgtttagtcatccttcctctgaatattatccaagttagtctttagttctgaaggtcatgaaaaataattttataatatttggtgccacttttatttgaagatgtcccagtgctggggatgactaatgtcagcattacaacatatgccatttttggttttatggcaaatggtattttggaacatgtagtttgatgtggggtacagtagaaagtgtttaatgatcattctactgtgcatctttaatttctgcccttggaaccacccagggtaagtgagatattcattctgaaagatctgaatcttcaattcattcatctataatttgatgaatgtacattcacaaaggttcataggttatcatgcaggatactttgttcccaaactgtgcttgcccttacatgtaagatatgtgtcttttgtaccaaaaattaagagaaaataagtcacttatgaaccattaaatgctgaactaagactcattcagtgagtgagtaactgcaaatactatgaacacagcctttcttacccctttttgaatagccccattgtctgtctatagaaagaaaaattactttataggtgtgtttgcaaaatcttgcctgtttcctgtttccaaaagttattgtattgagaattcctttgagaaaattcttgttgggatttatgtgttcagaagatgataattccttcatttaacagatatctattgtgtaccttctctgtgccaggctctgccctggcccgctaagaagatagcagcaaacaaaagaggctcattccctgcttacattcctacatgaggaaagaggacatgaaccagctattcagaaaagtatttaatgatctcagcacctaccttggggtcttcccaactggacattagaatcacttccatagggcccatgccagggttcagaaggttccaggaactaatatcccttataacaacccaataggcagagtttctagggtccccacaagaacaagcccagttgcaagaatcactactttaaagaagttcaaagctatggtaaacctaccagatgtttatagtttcttccaatttatgatacagtgtaccagtcagaggttatttttatcataagcaatgttgctggcattctacatttatcaagttactaggaaacagagccaggaattattttaaggtcaactttgtccttagagaaggaagagttgtgttaacactttacctataattactttcgtgagatgtatggaatgtgaagaatatttatgacctagactgtttatagctgatgccactgctatgcagtcattatgctacagactttaagtgatttttacatgggcatatgatgctgacaccctctttattttgcagataagtcatcatggtgaaaagccacataggcagttggatcctggttctctttgtggccatgtggagtgacgtgggcctctgcaagaagcgaccaaaacctggaggaggatggaacactggggggagccgatacccaggacagggcagtcctggaggcaaccgttatccacctcagggagggggtggctggggtcagccccatggaggtggctggggccagcctcatggaggtggctggggccagcctcatggaggtggctggggtcagccccatggtggtggctggggacagccacatggtggtggaggctggggtcaaggtggtacccacggtcaatggaacaaacccagtaagccaaaaaccaacatgaagcatgtggcaggagctgctgcagctggagcagtggtagggggccttggtggctacatgctgggaagtgccatgagcaggcctcttatacattttggcagtgactatgaggaccgttactatcgtgaaaacatgcaccgttaccccaaccaagtgtactacaggccagtggatcagtatagtaaccagaacaactttgtgcatgactgtgtcaacatcacagtcaaggaacacacagtcaccaccaccaccaagggggagaacttcaccgaaactgacatcaagatgatggagcgagtggtggagcaaatgtgcattacccagtaccagagagaatcccaggcttattaccaacgaggggcaagtgtgatcctcttctcttcccctcctgtgatcctcctcatctctttcctcatttttctcatagtaggataggggcaaccttcctgttttcattatcttcttaatctttaccaggttgggggagggagtatctacctgcagccccgtagtggtggtgtctcatttcttgcttctctctttgttacctgtatgctaatacccttggcgcttatagcactgggaaatgaagagcagacatgagatgctgtttattcaagtcccgttagctcagtatgctaatgccccatcttagcagtgattttgtagcaattttctcatttgtttcaagaacacgtgactacatttcccttttggaatagcatttctgccaagtctggaaggaggccacataatattcattcaaaaaaacaaaccggaaatccttagttcatagacccagggtccacctggttgagagcttgtgtcctgtgtctgcagagaactataaaggatattctgcattttgcaggttacatttgcaggtaacacagccagctattgcatcaagaatggatattcatgcaacctttgacttacgggtagaggacattttcacaaggaatgaacataatacgaaaggcttctgagactaaaaaattccaacatatgggagaggtgcccttggtggcagccttccattttgtatgtttaaagcaccttcaagtggtattcctttctttagtaacaaagtatagataattaagttaccttaatttaattaaactaccttctagacactgagagcaaatctgttgtttatctggaacccaggatgattttgacattgtttagagatgtgagagttgaactgtaaagaaagctgagtgctgaagaattgatgcttttgaactctagtgttggagaaaacttgagagtcccttggactgcaaggagatcaaattagtccatcctaaaggagatcagtcctgaatattcattggaaggactgatgctgaagctgaaactccaatactttggccacctgatgggaagaactgaaggcaggaggagaaggggatgacagaggatgagatggctggatggcatcatggattcaatggacatgagcttgagtaaactccaggagttggcaatcgacggagtcctggcatcctgcagtccatggtgtcgcagagttggacacgactgagtgactgaactgaggtgaacccagattttaacatagagaatgcagatataaaaactccatattcatttgattgaatcttttccttaaccagtgctagtgttggactggtaagattataacaacaaatataggttatgtgatgaagagaatagtgtacaaagaaaagaaatatgtgcatttctttattgctatcataattgtcaaaaaacaaaattaggtccttggtttctgtaaaattaacttttgaatcaacagggaggcatttaaagaaatatcttaaattagagacagtagaaatctgatacattcagagtggaaaaagaaattctattacgattatttaagaaggtaaaattatttcctgggttgttcagtattgtcacctagcagatagacactattgttctgcactgttattactggcttgcactttgtggtatcctatgtaaaaatacatatattgcatatgacagacttaagaatttctgttagagcaattaacatctgaactatctaatgcattacctgtttttgtaaggtactttttgtaaggtactaaggagacgtgggtttaatccctaggtcatgtaaatcccctggaggaggaaatagcaacccactccagtattcttgccaggagaatcccatgggcagaggagcctggcagggtgcagtccatgcatagggttgcaaagagtcagacaagacttgagctactaaacaataacaacaataaatgctgggttggctaaaaggttcattaggttttttttctgtaagatggctgtctttaacttcattcgaaacaattttgttagattgtatgtgacagctcttgtatcagcatgcatttgaaaaagaaaacaacttaccaaaattggtgaatttttgtatagccattttactattgaagatggaagaaaagaagcaaaattttcagcatatcatgctgtattatttcaagaaagataacacaaccaaaatgcgaaaatgtatttgtgcagtgtatggagaaggtgctgcaactgatcaagcttgtcaaagtagtttgtgaagttttgtgctggagatttcttactggacaatgctccacagtcgggtataccagttgaagttgatagtgatcaaattgagatattgagaacaatcaatgttataccacgtgggagatagctgacatactcaaaatatccaaatagaaccttgaaaaccatttgcaccatctcagttatgttaataactttgatgtttgagttccacataaattaagcaaaaaaaaaacaaaaacaaaaacacacaaccttgaccatatttgcatatgcagttctctactgaaatgaatgaaaacacttttgtttttaaaaacagattttgatgaacagtggatactatacaataacgtagaatggaaaagactgtggggtgagcaaaatgaaccagcaccaccaaaggccaggcttcatccaaagaagatgtgtgtatggtgggattggaaagtaatcctctattatgggattcttctggaaaaccaaaaaatcaattccaacaagtactgctcctaattagaccaactgaaagcagcattcaatgaaaagcatccagaattagtcaatagaaagcatataatcttccatcaggataacacaagactacatttctttgatgacccagcatggctgagaggttctgattcacctgctgtattcagacattgcatctttggatttccatttatttcagtctacagaattatcatcatgaaaaaaatttccattccctggaagattgtaaagtgcatctggaaaacttctttgctcaaaaagataaaaagttttgtgaacacagaattatgaagttgcctgaaaaacggcagaagatagtgactatgttgttcagtaaagttcttggtgcaaatgtgtcttttatttttatttaaacactaaaggcacgttttggccaacccaatactgaatacttaaaggaaactcttccgtgttgtccttagccttacagcgtgcactgaatagttttgtataagaatccagagtgatatttgaaatacgcatgtgcttatattttctatatttgtaactttgcatgtacttgttttgtgttaaaagtttataaatatttaatatctgactaaaattaaacaggagctaaaaggagtatcttccacggagtgtctggctgttttcaccagtgtgcacaccatgttggcagcttcatttggggggttaatatgagaaaagtggcacattcagtcctcacactgccagttgcggcaggagggcttctcctgatcctgcctcagccttactcccagtcacatgccagctgttctctgctaccttttcatatttttccatgaatacccgtcaaagttactactatagcggaggaaaacagtccttgcattctggaagattttttctgaccaggattttgaaatagaggattttcgtgattaagatgagacttaacaaagtatctaccttatgcctgtacccacccttgacaccatttcaggtcataaactgtgaggcctggtgacaacacccattgaattgaaattcaacactgtacggtcaatatggctactttcctttgttacaggctttcaaatggttcttcatatgtttcctccttcccaagtatgaggtgccagctcccagttttccttcacaaaggttttcttctgcaactgtagttcattaacagccggaagaaataataaatgatagtggttgaaatcataacatttattaacactttaataaatgccagtgtccttcagtatctgaacagaggatcaactttgcattaaaaatgaaaagattaaaaatcaacatcttgatatcccataattcacaaaataatttaaaaatgacataaaatcctcaaaagcattactcagttaatctttaacataagaagtgctaggactattttcatgctgtccttttggccatatgtaagattatttaaaaatagactattcattatctgccaatcataatctcccaagaataccccactgaaaagatgtcagttatacaaagcaaggtatttacagggccgaagtgaatgatacacatctgtatttttctcaggctaccatgttttcttcctgttacttccaattcctttgagttgtgctaaagaaatttctttatatttcatatgtatttttaaatagaggataattactttacaatattgtgatggtttctgccatacatcagcatgaatcagcatagggcttcccaggtggcactagcggtaaagaactcacctgccagtgcaggagacataagagatgtgggttcaatccctgagtcaggaagatcccctggaagagggcatggcaacccactccagtattcttgcctcgagaatctccatgggcagagcagcccggtgggccacagtccataaggttgcaaagagtcggacacaactgatgtgacttagcatgcatgcatacatatggccccttccctcttgaaccccctctaccacctccctccccacccacccctctaggttgtcgcagagtactagctttggtttccctgcatcatacattgaactctcactggctggctgttttacatatggtatatgtttcagtgctattctctcatatcatctcacactctccttcccttactgtgtccaaaatgtctgtgtttcctttgctgccctgcaagtaggactatctttctagattccatatatatgtgttaatgtatgatatttgtctttctctttctaacttatttcactctgtataataggctctaggttcatccacctcattagaacagactcaaatatgttcctttttatggctgagtaatattccattgtgtatatgtaccacaacttcattatccattcatctgtctatggttggacatctaggttgtttccatgtcctaggtattgtaaattgtgctgcaataaacattgaggtatatacatctttttcagttctggtttcctcagggtatatgcccagtagtgagactgctgggtcatatggtaactttgggcttcccttgtggctctgctggtaaagaatccacctgcaatgcgggagacctgggtttggtcctgggctgggaagaccccctggagaagggaatggctacccaccccagtattctggcctctagaattccatggactgtatagtccatggagttgcaaagagttgcacacgactgagcaactttcactcacctatggtaactttatttctagtcttttaaggaaactccatactgttctccatggtggctgtatcagtttgcattatgaccaacagtgtcagagagttcccttttctccacatcctctccagcatttattttttgtaaactttctgatgatggccattctgaccaatatgagatgacatctcattgtagttttgtttgcatttctctaaaatgagtgatgttgagtatcttttcatgtaattattagtcatctgtcatctttggagaaatgtctgtttgagtcttctgcccattttttaaatttggttgttttttgttactgagctgcttatgtattttggagattaattcctttcagttgtttcatttgctattattttctcccattctgagagttgtcttttcaccttgcttatggtttccttcattgtgaaaaaacttttaagtttaattaggtcccacttatttatttttgtttgtatttccattattctaggaagggggtcaaagaggatcttactattctgcctatgttttcctctaagagtcttatagtttctgatcttacatttaggtctttcatccattttgagtttatctttgtgtatggtgttaggaagtgttctaatttcattcttttacatgtagctgaccagttttcccagtaccagttattgaagaggctgtcttttctccattgtatatttttgcctcttttgtcaaagataaggtcctcatcagatcagatcagatcagatcagtcactcagtcatgtctgactctttgcgaccccatgaatcgcagcatgccaggcctccctgtccaacaccaactcccggagtttactgagactcacgtccatcgagtcactgatgccatccagccacctcatcctctgtcatccccttttcctcctgcccccaatccctcccagcatcagagtcttttccaatgagtcaactctttgcatgaggtggccaaaatattggagtttcagctttagcatcattccttccaaagaaatcccagggctgatgtccttcagaatggactggttggatctccttgcagtcggactctcaagagttctccaacaccacagttcaaaagcatcaattcttcagtgctcagccttcttcacagtccaactctcacatccatacatcaccacaggaaaaaccatagccttgactagatggaccttggttggcaatgtctctgcttttgaatatgctatctaggttggtcataactttccttccaaggagtaagcatcttttaatttcatggctgcagtcaccatctgcagtgattttggagcccagaaaaataaagtctgacactttccactgtttccccatctatttcccatgaagtaatgggaccggatgccatgatctttgttttctaaatgttgagctttaagccaactttttcactctccactttcactttcatcaagaggctttggtgcatggatttatctccaggctttctattttgttccattggtctatatttccatttctgtgacagtaccatactgtcttgatgaccatagctttgtagtatagtctgaagtcaggaaggttgattcctccagtgtcattcttctttctcaagattgctttggctatttggggtcttttgtgtttccatacaaattgtgaaagtatttgttctagttctgtgacaaataccattattagtttgataggaattgcattgaatctatagattgctttggataacatagtcattttcactatattgattcttccgatccaagaacatggtatatctctgagacaggaaacccgcgctgtgagtgcttgatcaagcccaagagaatagtccgcaagccggtttttgtgtgtttgtttttggccctttggtaactatcggtaaatttattcctaggtatttttgttgttgttgttgcaatggtgaatgggattgtttccataatttctctttctgatttttcattgttagtttataggaatgcaagggatttctgtgtattaattttatatcctgtgactttactgtattcattgattagctctagtaatttttatgtggcctctttatagagtttcctatatagaggatcacatgatctgcaaacagagttttactacttcttttccaatctggatccttgtgttaaaggatttttactaaaaaattaaaatatcaattttaaataactgagtctaactcttacagaaggtttttctggagaagtgtcaggtgtcaaactttctttcccttcctctctctctggaattaaagccaaagaagtgtcctctgccttggaagaaattttgggtctgtattgcttctcactctagtgggaaccttaatatggccagaacctgagcttccccaggctcaggccctgaccttccattggtctaagcaactgacctacatagttcatttccacttgagaatggtcagttcctctctggctctttgaaactcctggaggatttagcttctcctgcattaactggaggaactaaacccatcctttgccccactcctgtgaggcctacccctgttctccaagaagccacaccttctgctacacacattcagcctatgagcttcaactctgccttgctacaattttcctttcctggagagctggtgttctgttctttccctggagtagtgtgcctcaaacttgaatgtgcacctgacaggggcccaagattctgcatttcttacaggttcccagatgatgccatgctggttctgtgaaacttcactggaacagctccctcaggatttcacactggagcctctaccagcaccacctgaagttcaacacaagttgctgcaccccaccccagagtttctgattccagagtgcagggtaggaccagagaatttacatttctaacacactccctggcaatgctgctgttgatgtggagattgcaaatggagctccactgctctacaggaagatgtacatggaatagaaggcaacctggccctgaaaaatagagcagttaggagactaaaaatctaattggaatgctccctgaggaggagagagctgagagctctagggatgaaaagcaaaggagacataaggaagtagttaatacctgctgcctgaaaaactggaagcactggtgagtcctgaggcccaccactagtgagagattcagctaaacttggaatagtagccaggccacaaatgcagcacttctcaaattcagatgtgcgcacaaatcacccaagaaccctgtcaaaatgcagttctgaggccatatgtttgatgtaagcttggagatgtgtcatttctataagctcccaggtgatgtgtggtcccagtggtcccaggaccacaccaagaaacaaggacctagaagcctaagtcatctcttctaaccgtggccaagactttaaataagcattgaagtctcaggagctggggggaggtggggagtagccaatagagagtcttcaccttttcttgatttagccctaagttttgcctgtcgtgctttgagagcacattcctcttacctatcaacctcctgctggcagcagtgaagtcagcttgtgtattatctctgaaacaagctgaattagttggctgcccatgggaaatatcaaatccagagacactctgtcagtttttcaaggtcatacaaatagtgagtgaaaattttagttgctcagtcatgtctgattctttgcaaacttatggactatagctgccaggctcctctgtccatggaattctccaggcaagaatactggagtgggttgccataccctcctccaggggatcttctggacccggggatcttctggacccagggatcaaaccctctctctgttgcaggcagattctttactgtctgagccaccagggaagcccacacaaatagtatgttcaccaaagcacattgtggaaactctttgccttggtttgtgtttatatttaagggtttggctcaaaggtccgacatctcagtcactgtgcacaactcatggcctctgtcaagggtgccccctggtgcagggctccagcttgaggggactcagttgaatccaaggggaacctgaaggaagggtcagaaatcctaaaagcaaattcagcccaaaatgcctcctaccctatttgattcctccatcactcactgtcccatacacacttctctcatattatttcagaagtgacctgtagccagggcccatagattagtagccctctccaatcaaaccatagttccctaagccctagaacacatacatgtcacctcgtgccagagcccctaggctggaggccaccagggtaattgggactgggggcttctttctccctaactgtcctggcaaatctgcccctttcctccttctctaaaaacaaacagtaaacaaacaaaagcaagatcgttatcttaatctttatatcgagtaaaaataaaagttttcagtaactctattctttagcacccttactcaacctaatcatttaagaaaaccttacaggcccttgtttcattgcctttcttgttgaatataccatcttgattagttttctggggttgccattaaaaaaaaaaaaaagtgccacagattgaatggcttaaacaacagatatttactttctcactcttctggagactggaagtctgagattaaagtatcatcagggttggtttcttctctgacttgtagatggcctccctatgtcttcccacagtcttccctttttgtgtctctgtgtcctaatctctttttataaggaccagttcatgctctatcatgagaccctatggactatagccctctaggctcctctgtccatggggttttccagacaagaatactgtgggttgctattttctcctctaggcaatctttctgacccagggatcaagcccacgtgtcctgtatctcctgcattgcagatggattctttactgctgagccactggggaagcccttttataaggactgggcttcccttgtggctcggccggtaaagaatacacgtgcaatgcaggagaccagggtttgatccctgggtcaggaagatcccctggagaagggaatggtaacccactcctcaaattgtatttgaggtgaggactgctactgctgctgctaagtcgctttagtcgtgtccaactctgtgcgaccccatagacggcagcccatcaggctctcctgtccctgggattctccaggcaagaacactggagtgggttgccatttccttctccactgcatgaaagtgaaaagtgaaagtgaagtcaccactcctcaaattggatttgaggtaaggacaccactcctcaaattgaattagggctcaccctaatggcttcatcttaacctaactttaactctttaaaggccctaactccaaatacagtcattttgaggtattaaggactatgactccaacacctatcaagaaatgtcacagcagtatgttagtgtcagtctcaagagcgctcaaaggcagtcccaggactaagacaaccttaatggcagcctcacagtcacattctattcccttatcaggatcacactattccttcaatagactgagccactgcccatcaatccacttagaattgccaagggtacctatctcatagtgcccattgcagagcaaacagaaatgcttccattctggatacagaccctgaaacccagccaccatgcccccatggctcacacaaagagcttcataatcaaacaaatttgccctttggtttgtatccacacacaaatacactacaaacacacctggcttagagttacactgattatgagttaattgacataaaactgagtgttagctataatttaaggggtactatctctaattttcttgaagagatctctcatctttcccattctgttgttttcctctatttctttgcattggtccctgagaaaggctttcttatctcttcttgctattcttgggaactctgcattcagatgcttatatctttccttttctcctttgcttttcgcttctcttcttttcacagctatttgtaaggcctccccagacagccattttgcttttttgcatttcacgcaaagatgggcttgataaaggacagaaatggtatggacctaacagaagcaggagatattaagaagaggtggcaagaatacacagaagaactgtacaaaaaagatcttcatgacccagaaaatcacgatgatgtgatcactgacctagagccagacatcctggaatgtgaagtcaagtgggccttaggaagcatcactaccaacaaagctagtggaggtgatggaattc

SEQ ID NO: 2 - DNA, 23 bp, Artificial sequence
  23 bp insertion/deletion in the putative promoter of the bovine PRNP gene
tctcagatgtcttcccaacagca

SEQ ID NO: 3 - DNA, 12 bp, Artificial sequence
  12 bp insertion/deletion in intron 1 of the bovine PRNP gene
gggggccgcggc

SEQ ID NO: 4 - DNA, 14 bp, Artificial sequence
  14 bp insertion/deletion in the 3′ UTR of the bovine PRNP gene
tttttgtaaggtac

SEQ ID NO: 5 - DNA, 18 bp, Artificial sequence
  Forward primer PRNP47784F targeting the putative promoter polymorphism
gtgccagccatgtaagtg

SEQ ID NO: 6 - DNA, 17 bp, Artificial sequence
  Reverse primer PRNP47883R targeting the putative promoter polymorphism
tggacaggcacaatggg

SEQ ID NO: 7 - DNA, 19 bp, Artificial sequence
  Forward primer PRNP49686F targeting the intron 1 polymorphism
ttaccctcctggttaggag

SEQ ID NO: 8 - DNA, 19 bp, Artificial sequence
  Reverse primer PRNP49777R targeting the intron 1 polymorphism
ctagattcctacacaccac

SEQ ID NO: 9 - DNA, 22 bp, Artificial sequence
  Forward primer PRNP67976F targeting the 3′ UTR polymorphism
ctgttagagcaattaacatctg

SEQ ID NO: 10 - DNA, 20 bp, Artificial sequence
  Reverse primer PRNP68070R targeting the 3′ UTR polymorphism
ctagggattaaacccacgtc